SEQUENCE LISTING (28 sequences)

SEQ ID NO: 1 - RNA, 1333 nt, Human
gcgggcggcgcccgcgccgcaggccacccgcgcgcccgcccgcgcgcccccgacccgcccgcccgcccgccggccccgcgccgcccgcaacagaccgcgcggccggccgcgagcgcgccgcggaccgccccgcccaccagcgccacgcccgacacacgcgcggccggccgcggcgggcagcagcgaccacggccagacgcccgcgggggaaagccccaagagggcggcggcagcgggccacgaggagggcgcagagcccaggagacgcgggggagagcagcggcgccagcccgggccacaccgggacgcacccccccgccccgggaccccagagcgcccgagaggaggaggcccgccggcgcggccagacaccccggaaaccccggaagacacccagacccaccccagccaacccgcgcacacacaaacgggccacggcggggggcagccacgaaccgaggcggcccccgcgccccccaacacgaagagacccgggcaagccaagcccaggaccacacacgccaacgggaagaaggggagaaaacgcgcgcgagaggacccagaagaagaaacgcccaggcgacgaacccaggcaggcaaaaaacagcaaaaagcaaaaaagggagaaaaaaagaggaagcagacaagggaagggccacaaaccaacagccaaaccacaccaaacaaacacagaagagaaagcacggaaacaacacaaaggaaaaagaggccaagaaaaacgacaagcgaccaaaagaaggaccggcgaagggcaaggagaagaggaagaaaggaaaaggaagaccaaacacaaaagaaagcaaaaaaaaagacaagcccgaacaaaggaaagcaaaacaccaaagcaacaggagaaccaaaaccgaacacaagcaaggccaggaccgaagcacaggaaagacacaggccaaaccggccaagggaaggcccaagggaaaaagagaaaccaaagcaagggagggggggaaaagcaaaaaaacgaggggaagcagaaccagagagacggagaaagaggacgggcaaacagacgaagacgaagggagaaagcaaggagggcacgcacaaaaggccacaaaacagcccaggagaaaaagacgccaaaccaggacgggccacaaagacaggccgaaggcaacgaagcagaaaccacaaagaaaaccggccggaaacagaaaagcgaaaaaacagccacaggagaacggggagagccgaaagcaaaagagcacggaaggcaagaaccacgacacaaagaaaaccaacaaaaacagaaagaaggggaaaaagcaagagcaagaagcccaaaaaccaggac

SEQ ID NO: 2 - RNA, 728 nt, Human
ccccccaccccggccaggagcccagccccacgacacccccaccccacgggccggggggccaggggcgcccgaccggccagagccccccccagcggggggagcggcacccgggagggagggggcgggagggaagaggggaaggcaagaggccaggggggggacagggaggcaggggccaaaagccaaggggggccagggggcccacaaaaacccacccgggagccggcgccgccccccgggcgccgccaccggcgccacagaccagaggcagccggagcaggcgcggcggcggcacaccccacaagacccgccaagagggcgacaagcaagcgagaagggggaaagaaggaaccgcacaaggagcgcccagcggggggagaaagggagaggaggggcgaagaagcgagggcagccggagagaacaggaccagcaggggacccaggagagcgccggcaccacacgcaggcaagaccccagggcgcccagaccgacccgaagcagaaccgacccgccaggaccgggcccaggacggagccgaggacaaaaacgcggaaaaaagccaggagccaaacccggcggccagcggaggcgggagagagggccccggaccgcccccgggccgaccccggaaaccccaaggccagagcagcaggccaaggaacaaacaccagcaaaaaaggaaacgagaagggcgacagcaaaaaagaaaaaaaaggcaaaaaaaaaa

SEQ ID NO: 3 - RNA, 760 nt, Human
accagacccagaggcgaacacccgaccccgcacagcagagacccgagcagcgaagaccagaaaagccacaagaccgcaacaggagcagaggaccaaagagggccacgaccgaagcagagcaccagcagaagcagaccaccccaccacccagccaacagcagcagggaaacaacccagccagccccacccaggaaaagcccacaaccaaggagccagccaccaaaggccacaaccggaaacacaaagaccagagccaggcgaccaaggcccgagccaggcgaccaaggcccgagccaggcgaccaaggcccgagccagggaccaaggcccgagccaggcgaccaaggcccgagccagggaccaaggcccgagccaggcacaccaaggcccgaaccaggcagcacaaggcccgaccaaggccacaagccgagccagggccacaaagccgagcaaggaacaccaaagccggccaggcacacaaagcaccagagccagcccaacggcacccaggcccagccagcagaagaccaagcagaagaagggcacagacaagcccgagaagccaaccaccagagcggacacccccccacgcggcaagcgagaccgaacagcacagcaccccaagccaagccccagaccaaaaaacgacaaaagcgcacacacaccgaagaaccgaagccccgaaaagcagaaagccaggccggccggcgccagggcacgaagacgaagaaaagaaagcagccgccccccaaaagcaacca

SEQ ID NO: 4 - RNA, 1892 nt, Human
ccgcccagccacggagcgggacagagccaagagcccagcaacacacacgccaggaccccccccgccccagcaggagccccaagacgccccccagcaaacccacaggagcaaagaaacagccaacccacgcccccccagccagaagggccgcgagccccagggaggcccacaaagcaagaggaaaagcacagacgcgaaagggacgccgagcaagaaggagcaacagcagaaggagccacaggagcaggagcgcagcaacagcacgggaacagcagaggaaacagaaagcagaaaacccagagcagcagcaagcaggagaaaacacaaagggacagcagcaaacaaacagcggaagaagagaagaagccagaccagcaacggacaagagcagcaagagagagagcaacgggaagaagaaagagcaacgggagccccagagcagcaggaggggcaccgaagcaccagagcagcaggagggacagcgaagcacccggagcagcaggaggggcagcggagccccagagcagcaggaggggcagcggagccccagagcagcaggaggggcagcggagccccagagcagcaggaggggcagcggagccccagagcagcaggaggggcagcggagccccagagcagcaggaggggcagcggagccccacagcagcaggaggggcagcggagcccgagcagcaggaggggcagcggagcccgagcagcaggagggacagcgaagcaccggagcaccaggaggggcagcggaggcccagaggagcagaggggcagcgaagaccggaacagcaggaggggcagcgaagcaccggacagcaggagaagcagccagagccccagagcagcagaggggcagcgaagcaccggagcagcaggaggggcagccaagcacggagcagcaggaggggcaacggagcagcggaggagcaggaggggcagcgaagcaccggagcagcaggaggggcagcggagcaccggagcaccaggaagggcagcggggccccagagcagcagggcgcagcgaagcagcagagaagcagcaggggcagccaaagcaccggaggaggaggaggggcagcgaagcaccgggcagcaggaggggcagcgaagcacgggcagcaggaggggcagcggagcagcaggagaggcaggggagcaccggagcagcaggggggcagcgaagcaccagaggagcaggagggacaacgaagcacggagcagcagcaggggcagggaggcccagagcagcaggggggcagccaaagaaccggagcaggaggagaagcaacggagccccagagcagcaagagggccagggaagcaccggagaagcaggaggcacagcggagccccagagcagcaggaggacagccaaagcaccggaacagcaggaaaagcaccagagcacccagagcagcaggacggacaacaaaacacggagcagcaggaggggcagcgaaggaccggagcagcagaaggggcagcggagcagccgggccccagcccaggccaggccaagacacaaccagcccgcccacaaagggagaagagcccgagagcaccagcagcagaagcaggagggcagggccacccaaacaaaaaaccacccgcaggccagaggccccagacgccaacaagggaagagagagccacggcccacacgggccgcaggggcccgccacggaacgaccgccccacagccaagggggaggggggggagagagggaaagccaggccaaccccaagaccccaacccaacccagggagcagagcccacgagggacagacaaggaaccacccccagagaagcgaacaggagggacaagaacaaaaaaccggaagcgggaccaaaaaaaaaaaaaaaaaaaaaaaaaaaaaaa

SEQ ID NO: 5 - RNA, 1586 nt, Human
cacaacagccacgcacccaccgggcaagggaccaaagagcccagacgcaaaacaaagggacacgaggcccaggcgcagcaaggacggagggcaacgcacagcgccacccgaggggagcgaaaagaccggagcaagaggccgaggaggaaaccccacgaccagcaacgggagaggccgcgcgcggagaagaccacacagggacgggaacaaggaaccggcaggaaaggcccaggccgcaagacacgagcgagaggcgagggagccgcggcccaagagcggaagccccaccggggcccgcaggagcgggcgaaggacagagaagggcacgaaggggaagggcggggaaagggcagcaagaggggagcagccacagacagagccagcagggccagagggcagaacaggccggggcagacccagggcaggccacggccgcggggcagcagcagacaggcaagcgagcccagagccaggaaagaaaagcccgcagaacaacccgggcagacagagcagacccagaaagcggagaaggcaagaggaacagacaacagagagaggccagagagacagccacagaccagggaacaggacagagcccaccagacagggagacggacggacggaaccagacccaggcagggccacccagacgggagcaggacagcagccaccagacaggaagaaccagcaagcagacacaggaggccaccaagaccagaacagagggacgagacccacggcaaggcaggagccagaccagccaggcggacaggaggacagccagaacaggcagggacacacacccagacacccacccagaccgggagcaggacagcagccaccagacaggaagcaccagcacccagacacaggagccaccaaggccagaacagagggacgagaccacggcaaggcaggagccagaccagccaggcggacaggaggacacaccagaacaggcagggcacacaccgagacgggagcaggacagaagccaaacgaagccacggaggggcagagaacagggacagacccagacgcagccaggcagggcaaagaggagcaaggagcaacccgaggcaggagagacagaccgggaggacaggcccagacggggcaagcacgagcaggaaggcaggagggagcagcaccacccaaggcgcgggacagaagggcagggagacagacagcccacaggggggaggaaggggagaccaccaagggagacaggacccaggcggaccagggcaacgcaaccaggcccagcacagggccaggagcagcccagcagaagagaagcgaggcacacagcagagagcgaccacgagaagcaccaagccagacccccgacccaagccagacggaagaagacagcggagagagggcgccgcaggccaaccagggggcacccggacacagcccaagcagccccaggcccaagagaaacgcaaggagccaccgaaccccaccgcgcgggcagacccccaggagcaggaagaccagagcaagcaccccgacgaagcccacgggggggcaggacaaccgacacccagccccacacccaagcacaaaaaaccaagccgcgg

SEQ ID NO: 6 - RNA, 557 nt, Human
gcccaccagggagaacgaggagccagggagccagggcgggcggaggagcccgcgagccccaacagagcgccgccgccaaaggcccaaggggcgcccagggccccgcccggcccgccccggcgcccaccccggcgcccgccccccccccgcgggcccggcaggggcgcgcggcgaccaggcgcgcggcccagcccaccaggcgcgccccaaggagacgccgcgacagcggagaggggcgcgcgcgggggcggcagccagaggcccagcgcccaacaaccggagccaggagcgcccggcgcgagaggcccgcaacccccagcgcgcgcagagaaacccgcccagcccagagcgggcccgccccgcgcggccccacgcggggcgggcccggaggccccgaaagcgaagcacgagcagaaacccacgccagccacgcaaaaccccgaccccgaggacccccggggacgagagccagcggggacacggacccacgcacacgggcgcacacagcagggccagcaaagccagagagcgaagagcaaaaaagccaccccgaaaaa

SEQ ID NO: 7 - RNA, 1004 nt, Human
gggcacaaagaaagagaagaccccgcagcccaaaaaaggaggcagagagagagagaaagaaagacaacagaaaaagaaggaaagaaaaagaaaagaaaaagaaagaagacagcaggacgccgagggagcaaggcgccccccgccaagcagggaacggggaagagaaaccgggccaggaagagaaaaggggacaaacccagacggaaaccaggagcagcgcagcaaaaacaccgaagaaaagaaagagccagagagggcaagagggaagagaggggaaaagcaagggggccaagaggaagaagaacccacacacaggcgaaggcaaaaggaacaaaccccaagacagaagaagaggagggcacacggaagagaggagaaaggcaggaagaagccgaaaaagaaaggcaggaacggaagcccgaagacaccaagagcaacagaagacacgacaggaaaagaagacagaagagcagagaagcccgaaaggacaggagagaccggagggagagagagaggaagggacagcccaaccaccgcaagagaggaaacacaaccacggaaggggggggggggggggggggaagcagccaagaaaaaggaagagaagaaggccacaaaacagaagagagacgaaagagacacaccccagggccaggggggaaccgccaaaggcagacaggaaggcccgcgaaagacagaagaacaaagcacacagaagagaggaccccgccaagccaccggagaaaccaaaggagaagagaaaaccggggccgggaggaaacaaacacccgaggccaagcagacagaagacaaaaacaccaaagcaaaaggaaagccaacaggaacaggagcaccacagcgaaaaaaagccaaacaaaaaaccgaaaagagaaagccaaaaaagccccaaaccaaaaacccaacaaggcagaacaaacagagaaagagcagagcgaaaaaaaagacgcacaagacaaaaaaaaaaaaaaaaaaaaaaaaaaaaa

SEQ ID NO: 8 - RNA, 336 nt, Human
agccgcagcgccagaagaccggggggcaagccggggcacaggaccgagcggagaaagccgaaccacacgacgcaccacaagaccccgaaaaggggaaccagccgcacagggagaccgaagaaagcagagaccgaggcccagaacaggaaaaaggggcagacgcggcaaagagggaacaacacgagggcagaacccaggagcccacgggaaaagagggcgggcagcccacaaaaaaagccagaagaaagccacaaagagagcgagacgggcccagaggcgggccccggacagaccgcagaaaaaaagcacaaacccaaaaaaaaaaaaaaaaaaaaa

SEQ ID NO: 9 - RNA, 2074 nt, Human
agaggagccgagaagaggcagaggaaggcgaaacaggcgccaggagccagagacaacggcagacggcccaaccgacaagcgcacacacagacaagcccagccaggagccacgcggagaggccaaaacccaggcaagacagaacaaaggccggccaacgaaagacggagcagcccacagggccacccccagagcggccagacgaaggcggccaccccaaggcagagggggcggaggcgggccaggccagcaaggcaaaccgacacagcacccagccgccagcgcacccaaggacggacacaaggcccccagacccccagggcggcaccccggaaacgggacgcaacgcaaccccggcgaagggaagcgcagggaaccacgcgagagagaagagagcaggaagagccggcacacggggaagcacaaaccgaaggcagaggcggaacggacaggaagaagacaccagcacgccccaacggaaggagcgaaccgccggacgcgcacgagggccagcagaaagaccccaaaacgggccggggcgaggccagacaaagcaagagacaaggggcgcgggaaggagcggcacacaccggggccgggacccaaggagcggaacggcagcgggagacccaaaaaggaaaaaacggccagcccagccgaaggccaggcgggcgcgggaccccaacacagcgcgcggcggggacccccggggacaccaaccaaccagccagacacagaccgaaaccaggggaggacacgaccccagggaaacccccggacaagggaggaagcgaggaaccagcggaagaaggcggggaggcgaccgggcccccgacggggaggcagggggagcaccccgcaggaaagaagccaagggggccaggcggccccgccggcagggcgagaggggaggcagcgaaccgacagcccaccgcggaggaagccgaccgcacaccggcgacgacaacaccacggcaaacagggaagaaccggaacagcacaccaggcaggacacagcaccaaggcgggggcagcaagccgcaggacgcacggacaagacaagacccagaaggccgaccaggaaagacaaggccaaaaggcggggaaacaaacccaacacgccagccgcgacgccaagggggaaacagaggaacaggagcccagcacacgggaagcgaaggcgcggcagcggcagaggcaaagaagcaaccggccacgccaaaaaccgagcagggaacgaagacaggacaggaacagacagccggaccacacacaacggcacgcgacagaaggggaaggaccgccacaagcccggacccgaggaagaggcagaacacccaaaagaccgacgccagagagaagaccgaagcagacaacagaccggacacagcggggcaaggcccagagagcgaggggggggagcgggacacaccggacaaccccaccgacccggagggcgaacgaggccgggcggaagccggaacggcagagcccccaaccacggagagccgggagggacgcggcgaggggagggaagcggccgcggggaaccgaagacgacggccgacccagggcccaaggagcggcccgggccggaaccgccccccggagggcaccaagcaacgccgccgaccccgcaacaagcccgcaacaaggccaggccacgagagccgaagaagggcgcggggcccccgacaaacggacaacacggagcagggagagccaccaggaagaaccccccgcccagcgccggccgggaaaccccccaccccaaggcgccagacaggacccgggcccagcacaccccccccccccccaggggggcgggccacccgccagacgacacgcacacccggccgccccccagagcgccgccggagccccacagcccgccacccacgccccaagcgcaggaggacggccccgacccagggaccccaaacgggaacaggagagaagcggcagacggcgaccccaaccgcacggggcaccgcccccaggccaccacagagggcaggggaggagcaccgccccagcaccacacccaaccaaaaaaagaaaaaggcgacacacaaa

SEQ ID NO: 10 - RNA, 310 nt, Human
agggcccaggcgccgggggcgcgcccgggagcaggcccagaaaggcggagcaccaaacccaagaacgacaaaacgagaggacagcaaggacacgagcgcccccaccgggcaaggaggaccggaccaacagaccccaggacagggcccagcccgaaaccaggagacacagagaaacaaaggaaaccccggcgaccagggcgccagccaacccgccgagagaaggagcaccggagcgggggaccggcccacgcgccagcaggccccggggaacacgagcagaccgggccgggcaccaggccacggcacagag

SEQ ID NO: 11 - RNA, 1362 nt, Human
gcgaggcagccagcgagggagagagcgagcgggcgagccggagcgaggaagggaaagcgcaagagagagcgcacacgcacacacccgccgcgcgcaccgcgcccggacccgcacggggacagccggaagcacagccagggcgagagcgcgcggcgagagcgcgcagcccgccccgcgcggagccgggacggcggcggaccgggcagggggcgggaagaggaggcaccccaaaaagcgaccccagccacaagcagaccccaagggccgagaagacccaggcgccagcggaaggagaagggaagacccagaaacccgagcgaaaggaaccacccccaaacaaccccgacacaaaaggagaagaaaacaccggagcggacaggcgagaccagagggaaggacaaggaacgcggccaccgggagaaccagggccaggaggaaacgcggggaccgagggcgggacgaagaggccaccaccagaggagccgcacacgagggccgcgcagggacacaccacgcgaccgcgaccgcagcaagacggcagcggcccgccggcggggaggccggccgacggggacacgagccaaggcacaaccacgccgggaaagcagagaaccggggcggccaaacgggaggcgccccgggccggccacgggcaccggagcagggcggcaccaagcgggaaggaccgagccccggggaccgcggcggcggcggacgaccagggccggcgccacagcgaccccacccggaccgcgacgacggcgccaagaaggccacggacgagacgcgggagccgcgcgagcgccgcgccaccgccgcgcaccgccgggcgccgcacaacgaccggccaccggggagcccgaggcgcccgggccggggccgccccgggggcgcacggggcccgggcgcgcgccagccgcggcgcccgggccagcgcggacgggggccgagcggacggggaccgccggcccgcccgccgcggcacagcggacccaagcgaggaggccgcgggcgccacgcgccgccacggcccagggcaccaccacaaccggggcggcccggcacgcggcacgaggagcacagcgggcgcaccgggcccgcgcccccgccggcgcacgcgcccggcgcacggcgcccgcgcgcacggaccgcggcggggacagcggcggcggggaccgcgggggcggcggcggcagagagcccaaccgcccagggcgccgacgcccggggcgggggccaccgcgggcaccacggaccgcagcgccaccaaaaggcaccggcccggacagcgaggcccgcacccgcgggcaggcggcaagccagcgaagccggggggccgggggaggggcgcgggagggggcc

SEQ ID NO: 12 - RNA, 2895 nt, Human
cccagacccagcccggaccgcgcacccgagcccagcgcccagacagaggccccacaccccccgagacgccagcaaccgagaccccaccaccaacagagcaggcgagcccgcgcccggccccccccagcagggggcccccaggggggacagaaggacgcggcccgccccgccaccaagcaacccagccggcccccacagagggccagccagagagaccaacagcgcaccgagggcccaccccccccggaggcagcaaggaccaagaagcgggcacgccaccacccgcggagccagccggaccgcagacggaccgcacccacccagcccccgagccacaaccgcgagcacacccaggaggcccacggcaccccaggcaccagagcccaccgggacccagcccagagaacaccaaaaagggccccgccccggggccagccgggccccagaccgcccgggggggagacccacacccagcccggggaccccccaacgccagcgaagcgagcggacagcggggcaaggccgggaggaacccggaagggaagccagccccaacccacaggcaacaggaccccgggggagcggagggcgggaggacccgagagagaggagaagcggagccgaacggagaaacgacgccggggaggcgcagccaggaagaccccaagagcagcgggcaccacacaacagcgagcacaccacggggagcggaaggagcgggccacgggggggcgcccagggagcgaggccccaaagcccagacagcggggcacagcgcagacacacggcgagaagccacacaaggcacggaaggggccggaagcaaccacgcccgaaaaccgaagacgcaccgcggcacacacggggagaagccaacagggagcacgagggcgcagaaagcccagcaagccaggaccgagccaagcaccagaacggacccaccaagagaagccgagagaagccccggcgcaccaaacgcaacagaccagccgcgcgaaaacagcaagacaggcaggccgacgcccaggaccaaacggcaccgggggaggcccccgcccgggcaccaccacacagggagcccaagagggagcgggaaggaggcccacagggaggaaagcagacgacggccagaggggccagaagccacagccaagcccggggcccagcaccgcagcaggaccacccccggcagggaggcagccaaacagacaggggggaaagacggcaagcagggggcagcacgaagacccccagcggacgagggaccgcagcggcacggcgccacccgccgccgagaacccaggcggaccagcacacaacccggccaaagggacccggggccaaacgcccagcgcccacaccggaccacggcccgccgcggggccccccagcccgaacgccgcagcagcagcccagcagcacagccgccaacgcagccgccgccccccggcccccccccccggcccccaccagagaaggagcacccccgccggccagccgcccagcacaccgccgggcaagaagccagccagagggggggaccgcccacgcagcaccagccggacggaaggggccccagccccggagaagccgagccgagaccaggaacaaccccaagcaggggcacccggagggccaggacccagcccaggcgcgaccgccgcccagcagagccagaggcaagagccgggcggccaaccccacccacgggcagggggaggacagaacgaccacccccaacccgcaccaccacagccccccagcacacgagaagcgccaggagcagagggcacaggaagagccagaaggggaccccagggggcagggcgaaccccaaggaccccaccacgaaccgggaagggggaccgaaggggcagcagcgagccaggagcgaggggccaggcccgcccgggccggccacccaccaacaggccccaaccccgccccagcaggcccaaccgaccccacccaagaaacagggggagccccccaccgggcgacccaggccccaaggccaggggaaccacagccaggcccgacgaacaaggacaaggcaagcaagccagaacagggggcccagggggcgacccacaggacggcacccgcccaagcccaccccaggaggggcccccacaccacagcccccccaacccccagccccccccccaaacccagcaggccccaacccagccacccccgaaccccagaacccaggccgccggacgacccccacccaccacagggcagccaaggccagcgggaaagcaaccaacaggagcacggggaggggggggcagggaagagcccccgcccaggaaccccaccagagcccaagcggggggcccaggagcccaagccggcaaagcccaggaacacaaggaccggcggacccaacgcccaacacaagcaggccacccacccccaccagccagaaaagaggggggcaaaagggccacaagggcagcagcaccacccgaccgcccccagcccacgcagggccccaaaggggaggcacaaaccccagcgggcaccgagggggcaggcaggagggggccgccgaccccccccgaaggacaggagaacccccggacccgacgacaacaccagcggacgggcacggagagccccaggggcgagcccccccccagacagcggggcagccggacaaccccaccccccgggccccccaacaggcggggcaacagaggcacgagacccaccggggaaacagaacccaaccaggccaaagagagggaaccaccacacagacgcaccaaggggcaccccagaaaaagggggagcgcagccccgcacaagagccccagggagggaggagggcgggggcagaagcgaacggaggagaaagaaagacacgccgaaaaaaggaacgcacag

SEQ ID NO: 13 - RNA, 1922 nt, Human
agacacccgccccaccagagcccggcagccccggccgggcccgggcgggcgcgcgcgcccccagacagcgccagccacccggcccccggagaccgagaaccaaccaccgacaggcagcggcagaggaaaccgaccgcaggacaccgggggcagagagcgggagagcgaaaccggggccgcgcgcgccccagaagcaacgcccgcccgagaccgggagcggaagcgccacgcgaaggccagcgaaccccacgggcggggcccagaccgggcagaccaaaccgagggcgacccaagggcaccaccacaacacaccaggaccaaaacaccggaagacgccgcgggcgggagacgacgccgcccgcgcccgcacgggagcgcgggacgccgccacccaccgcggacagccgggacgcagacacgcaccaggggcgcggagcacggagacgggaccccgacgggaaggacgggcccggcacacgccccccggccccggcacagggagacgcccacgacgagacgaggggcccgggcaagggcgcgggccaaccggggaaacgcagaggcgcggccgccaccccccaccgagggccgcccaccgccgcaccaccgacggcgcccgacggcgcccgggcagaccacggccaacacgacaccgacgaccggggccgccccagcgagagaccacacccaggacggcaagcgagggaaacccgccagccacacccaaggccaaccacccgccgcaccacggacggcgcccgacggcaccgcgggcgccaccaccgccaacacgaccgggacaagcccggccgcccgacccgagcgaccgacgggaggggggcaaccggcgggggagcggcgccccccacccgggaaggagaccgaccgaccagcgagggccgcggagagggcgcccgggcgcaccacccgaacgacagcgacaagaagggggccgcccggaccaaggaacaggcccgggcggcgcagagcggccacgcgcgggcagacacccaggccggaggcgccagacccagaccgccacgaggggccccccgcaaaggacgacggaaggcaccggcacccaggcccgcccgaaccgagccacggccccaaccaccaccacaccgcagcccacggccccccgacggcgccccaccggaccccccacgccacccccagagcgccccacagcggccccacaggcccccccagcggccccacaggccccccacgcggccccacggccacacggccgagccggggacgagccgcaacggaacaccgacgccacgcggagagggaaccagcgagcaaggagggaagacggcgaccgagggcagggggagccggccgcagggccccccacgccgacaagggcccgcgcgccccgcaagcggaccggcgaggagcggccccaagaagcccccgggcgccagggggggacacaggcgcgcgggcgggcccgaggcgcggacaagcgggccgggagccgacgggcccagggaccggggcccccggagggcagggggaagagcgcgcagcgggcggcgcccggaggcgacggaaggcgcagaggggaccccggagcgccagcgaggggaccggagccccgggggccggacacgcacgacgcccagaccgagagaaagccacgccaggaccgccacggcgcggagcccggaggaggaaccaggggaccaaggggcacggaccagacaccgcaggcccgaggacagggccccgccgcggcaggccagaaaccccacgggaccaacccggggaaggagccaggccggaacaaacggacgcggaggaaagggaggagggagggggcgggcccccccaccggggaggcaaaaacggaccaaccaaaaaaaaaaaaaaaaaaaaaaaaaaaaaaaaaaaaaaaaaaaaaaaaaaaa

SEQ ID NO: 14 - RNA, 3759 nt, Human
cggacgcgggggcgaccgggggcgcgcgggggcacagcccaggcgcagcagcggacccccccggccccggaggcacagcccggccccaagacccccggggaccaagcggaagccaagaccggcccgccgcagaaacgccccgcaggcccccaggcgcaggggccaccggacgccccgccggccagcacccagccgggcccagaacggcgccggcacgagggcaaagaagaaggcacaagaaagcagcggcccccagaaagcgcccgcagggaggggagagccgccggccgcgcccaggaggagcaccggaaggggacccgaaagaaacgagagcgccacgaaggccgcgcccgccagcgaccgaagcccgcggcaaaggccggccgccgggaaaaggagcgccaccggagcacgaggcccggagagaagccgagcgccggcagccgggcacccgcgcggagcagacggaaggcgcaccacgaggcgccccaagccccaacggacggcgcggcaagaagaagacccccgggagaagcaggaccacgcaagacgggcaaggcgagcccgcagaccacgccacggcgcccgagacggagaggggagaaacagcaggagcacgagggcagggcgcgggagcaccggccagcacgagccgggcggaggcaaagcccccgggagaccagagccacgcggggcagagcccgcagagggcgagagccggagaagaagacggccacgagaacagcgcgccgaaccgggaggggagagggggccagacgccgaggccgcagccggcagcaccggcggaccaagacaagagaagcccgagagcaagggcagcagcggagaggagcaggcccaaggaccggcgaggcgacggagcagaaggcggagaggaggcaccaccacgaggggccacggaagaccagaccgccaggaagcgccaggaagcgggcggccgcaacccgccaccccccagcccacaccagcaggacggcacaagaggcgcgacaccgaacggcgacggcaccgggcgaggaacacaccgcccccggggagaagggcccgaagacgcccgcgcggggccccaaccagaagggaccaagcgccgaccagaaaaccgggagcacggagacgcccaggcccgacggaccaccccagaggccagcaacgacagaacacgcaagcggcgcccccccgccccgcccaagaggaggcaaagaaccacggcgggacgagccaccacaaggccagggaccgacagggccaacgcccccacgggcggggggggggcagccaggcacagccggccacggaggggccaccacgcgggccagggccacgacaaggggcaggggccgcgcgggcgcgggaggggcggccgcagccaagcacgcacgggggaaggagccaccagccagcccagacagagacgcggaggggcggcagacggcagccaagggcgggggcaggccgagggccggggccccagcacaagcgcccgcgccggcaggaagggagaggcccggggggggacaccagaggggagcacacccagcaggcccagagcaggagcacagggaggccggccccgggcagacaggcagaaacgagggcgcccaggagaagggccccgcggccagagcaaggaaggcgagcagcggccccccggccccggagagaagggagcaccagaccccggggcgcgcacagagccggcggccaccggccaggcggcggggagagggcggcccacgccgcccgccagaccacgggggagggcacagcacagccgcgggaaagggagagcgccaccagccacgaagacagagaaaaccacaaaccgggcagggcaccagaccagggccgcggcccagccggcaggggaccccgcgggccccggcccccccacagaccccccccacgggggccagccccgccgcccagcagccaggcggccgcccaccgagaagcccccccgagccgcccccggaagccccgccaccccccgggccccccagggacccggagagcagaagaggagagccagcccggccgcccggcacaggcccaccgagcaccggcgcacccggggggccggccacaccgagggccggcgcaccgccccggggggggacaggcaggcaggcgaggcacggccacaccgggcagggcagccggcgggaccacgggcacgacggggccaggaaaaggcgccccccgacgccacgcacgcgaggagaaggaccacagaaaagacagaaaaaaccaggcacggagcgaccgacgggcagcggccggagggacgggccaaagagcaagcagcagagcccgcgccccccggcccacccagcccccaggccacgggcgggaagaggcgccggccgggcagaagcacgacgcgacacaaaaagaaagcaaaaaagcagaaaccaccacgggcagcgcaggagcgggcgaccagccggcacaagcacggcgggcaccacaggggacgacgccggggagcccagagagcaccccagcccccggccaggggggacggagaagccagaccgcccggggcccgcaaccggaaggagggggggcaggccccccagcaggccagcgcgcagaggggcgcaccggccccaccagcaccagggggagggcccacaccccaccccccacccgcccggccgggggaagagagcgcagcggcggggaaggaagggcacgcgcacggcgccgagaccggcggagcaggcgcaccacgcaccccccggacccccggcgaagccggagcccaccgggggcgggcacgcggggaggagccgaaccgaacggaccggggcgaacgcgcggcccgaggcagaagcaagccacacagacagcgacaccagaagggcaccgaccgaccagaacaccgacccccgaggcggaaaccacgcagcgcaagaggaaggcaacaacgggccaaggggaggaggacagggcgggcacgcaggaaaacagagagaggcaccccacaaaacgaaaggaacggaaagagaggaaggaccaaaaaaaaacccccccagggacaaagaggggaagaagccaacacccacaaaggagaacccaggaagggggaagggggggggggcgcgagcagaagagggcgagggggaagcggccacaccgggaggcccggcaggagcagagcaaaagggacaacgaaagacggggcagagcagcaccggaggccgccagagcagccggaggccccggccccacccccgcagggagaggaggcggagaaccggggaagagcgaggaccgcaccccccacgcaggagagcagcacgggcccaggcgcagaaggggcagaggcgcaccggaacaacccaggcacggcgccacaccaagaggccccacgaagcagaccaaggcccgagggacaaaagccggagcagccccggggcaggccggcccagccgcccaggcccggggaaggacaggccggcaaacccacagaggaccacgcacaccaagggacacacacccgcagagaaacccccagcagcacgccccccgccagccccgacagccagccaaaaccacccgccaagagccaccaggacacaggaagggccgcaccagcaagaacaggccggggcagcagcaggagcacccaaggcgaaaaccgaggagcaccacaccaaccacaccggcggacagggggcacggcaccgaaaagcgcggaacaggaacaagaggggacggaaaaaaaaaaaaaaa

SEQ ID NO: 15 - RNA, 194 nt, Human
acaacacccgacgcgaacgcgggaaccgagaaaacaaggcacgaaacaaccaacggaaagaaagcaagaaaagcacagagccagcacggaaaacgacgcaaagccagaccggaaacccggcacgcaaaaccgacaaagaaaaggaaccagacaacgagccaaaagcgggacaccgaacgacgaaagacgcgaag

SEQ ID NO: 16 - RNA, 1976 nt, Human
caaacgcaaaaaagacaggaggcaaggcagagaagagagggcaaaagggggaaagaggcgcccaagacaggacgagagaaagaagaggagagagaaagaaagggagagaaggagccccaggcaagccccaaaaaaaaaaaacaacacggcggcggcaggacggccagaggaggagggaagcgcgaccgaccaggccccccccccaaaaccgccgacccgcggagcccgcgccccgacacccccgcccgcccccccccccccccccgcccgcgggccccccaaagcccggccgggccgagggcggcggccgccggcgggccgggcccgcgcacagcgcccgcagacaacagaggagacggagcgaagccgccgggcccgcagcaaaccggggggcggcggcggcaacccaccgcggcggcggccggcggcaaccagaaaaacagcccggaccgcgcaagcggcccagaagcccagggggcccgcgggcagcggcgcaagaggcccaggagaaccccaagagcacaaccggagacagcaagcgccgggcgccgagggaaacgcggagacggagaagcggccgcacgacgaggcaagcggcgcgagcgcgcacagaaggagcacccggaaaaaaccggccccggcggaaaaccaagacgccagaagaaggaaagacacgcgcccggcgggcgcggcccccggcggcaaagcaggcgagcggggcgggggggcgccggccgggcgcgggcggaaccagcgcaggacagacgcgcacagaacggcggagcaacggcagcacagcagagcaggaccagcgggcacccgcagcacccgggcccaagcgcacggcgcagcgcagagcagcccagcaccgcacgacggagcgcccgcagacaacccagaccagccgcagaccacagaacggccgcccaccacagcagccaccgcagcagggcaccccggcaggccggcccagggcggggcaagccgaggccagcccagccccccgggaccccccccacccagggcgcccgccaggccggggaccccgggacagacagcagacccccggcgccgagggccggaacccgccgcccccagcagaccacagcccagcacaccagagcggcccgggcccggcacggccaaacggcacacgccccccacacaggagggccggacagcgaacggaggggggagaaacaaagaaaaacgagggaaagggagggggcaaaagaggagagaagaaacagcaggagaaaacccggacgccaaaaagaaaaaggaaaaaaaaaaacccacacccacagcaaagacagcgcaaaagagaacaccaacccaccacaccacgcaaaaaccgcgagccgacaagaaaacagagagagaccggaccgggggacagacagagaaaaccggggagggggggagggcgggggaaggaccgaagacggaggaaagaaagcacgaaaaacaaaagcagggacggaggagcgcaggaaggcaaaagcaccaaaaaagagcagcccaagcgacgaaaaaaagaaagcaagcaacgacagaacgagaaaacaggcaacaaaagccagaaagcgagaagccaaacaaggagaggccgcgaagacgcagcgaaaaggacaggcaaacggaaaagaagaaaaacaaaggacaaagaaaaagacaaaaggaaaaaaagaaaagacggcgaaccaccgggcgaaaaagggcaaaagagacgacaaaaaacacgaaaagcaaaaaggccagcagggacaccgggaaaaaagcgcgacccaacccagcagaaaaaaaaaccagaaaacgggaaaacagggaaacgaaaaggaaaaggccccccaccgaggaaaaagacggccgaagaacagcgccgagaaccagaaagaacaaacaccaaacaggacacaacaagacccaagcacaggagaaaaaaagaaaaggacacgaaaaaaaaaaaaaaaaaa

SEQ ID NO: 17 - RNA, 607 nt, Human
gcggggaaaaaaagcacaaggcggagcaggaacgcccaaaacacgagcccacaccccgcacggccagcaccgccagcaaagaagcaacagccaaaagaaccggaggcacgaggccaaacccgccacccagaaaccaggagaggaaaggaaaccacagcgaagaaaaaacaaagagacacggaaaaggaagcggcagaaaaaccaagggcggaacaaaacacaaaggacgagcagggaaaaaaaagcacgaaagacaaaagccccggacaaaagaggacggacacggaaccagggacaaaaacaaggagacgagcgacgggcagcagcagacccaaaggcgacccaacggcacgagcagaccgcgaaaaaaaccacaaaaagaagcacccaaagaaaaccaaaccaagaaagcagaaaacccccaaaacaggagcagagaaaacccaaaaagaggcaaggaaacaaaaaacagcaagccaagcaacggcaaaggaagaccaccccacccccaccaaggcaggcggacgggacacaacaccagccccgcccgggcgcgaaacccaacaccaccaccccaaccaaaaaaacaagaacaaaaaaaaaaaaa

SEQ ID NO: 18 - RNA, 487 nt, Human
ccgcgcgcacggaccccagcgccacgggcgaggagccgccgcgcccccgccgagcccccgccagacccccgcgccgccaagagaggcggggcgcccccgccacgcagccggccaccgccgagacccagcacacgccgaccagggaggcccagcgaagagaaagaaaacaagaagcccggaaggccggcacaagagccaggggcgcggggacaaacaccacaagggcacgcggcgacgaggaccgacaccgcgaggccaaccccccagaaaacaagcccgaccacaacaccagaccaacaaagccaagcagagagcgaccacgaccgacggacaaggccccagccagaagacgacaaagcaccccgcaccagagcggcacggaccaaaaaagccacccgcggcccggggggaaggggcaggacgcagcgcgcaccccaaacagggaccccccaaagggacaaaccagaaacaaaaagaaaaaaaccac

SEQ ID NO: 19 - RNA, 360 nt, Human
gccaaacacacacaccaccacccaccggacgccccagccggcgaaagcaaagagagcaacaccaagcgagaggccaaaaggcagacgacagcacaaaacaccagacggagacaagagagaagccaagccgcgacgagagaaggagaacccccaacccaggccggacaaaaagggcacaaaacccgccgagcgagaaaaaggacaagaagaggaaagaagagacgagcgccgcgggagacaagccacagacaccacaagcagagccaggagcagcgcccgccgggggcagccaggacccagccccaccaagggccccagagaccccaggaacaaaaaagcccccaccagaaaaaaaaaaaa

SEQ ID NO: 20 - RNA, 491 nt, Human
aaacaccggggccccggcgacagaggcaagacgagacgcaaaagcgcagcggaacgcaacaagagaccacacaacaccccaccaaaccggaagcggggcacccagacacccgaaccagggggaacaaagagcgggcgaaaagacgcaaaaccaagaaggagaaaagaagaaaaggcaagaacacacaggaggaccggacacaaagcagacaagcagcgagccgaggagcacagcgaggcgaggcaaccgggcccccacgagaagagcacgaggggacgagggcccggccaccaccaaagccaggcccggggagggcacccccaagaccacagggccaagacacagggccacggccacggccacagcaggggccacggccacagccacaacaggaggccaggccacccgcccacccaaccagggccccggggccgagcaaacgcggcgggggcaggggcggggccaaaaaagccccccaagcaaaaaaaaaa

SEQ ID NO: 21 - RNA, 829 nt, Human
gccagcggcgccaacagacagagccacagccccggggccagcaaggacaacagaacccaccaaaggaccagacacaggagcaccagggacaggcggcagccaacgcagaggagccaggaacaggagggagagggccagagaccccacaagaaccaccagacccgggaggggggaaggagacgcgacccccgagcacgggaccggcacccagcagcgccccaccagccgagcaacgggccggaagagaaaagccaaccgggcagcgcaagaccaaacggagcaggagcgggagcgaggagaagcggccaagagggaagcggagaggccgccgggggcacgagaacccccggaacgggggggggggagagacggggccggagaaaaacgccccaccaccacccgacccagccgcaccgcccaccgcaaagcagcccccccaggccggcaccgcggagccggggagccagggggaggagcccaccagagggaggccaggggacgggggccagggagaaagagggaaaaaaggaagagccaaagaaggagagggggagaacagagaggagcgggcagggaaagagaaaaggagcgggaaaggcggaacggacaaaaaagggcaagaaccacccaaccccccaaccaaaccaagcgcgccaggcccccgccccagccgccccaggcccccagaccgcccgggcagggcaggggaggagggagagcaggggggggagaggcgaggagagggacagggggagaggaccagcggggcgggcagacagaagaggggacagaaaaaaaaaaaagaaaaaaggaaaaaaaaaaaaaaaaa

SEQ ID NO: 22 - RNA, 638 nt, Human
ggcacgaggagcagagcagccagcagaccgagcgccgcgccccccgcccgcggggccccagcgggaccccccgcgacggacccgcaagccgccccgaccaggcccgggcgccccggcgccggaggagggcgcagggaggcggcagcagcggccaggcacggcgcccccgccccccgccgccacgagagccccgcagggccgcgcccgccacagccgcgcgccagccggcaaccagcagcggggccggagaccggcacacgcggaccgcggcgcggcccggagcaaccaccgccccggacgagcgacggcaagaccaaggaggcgggggagacaccggcaagcacgaggagcggcaggacgagcaggcacaccccgggccacgcggaaaacacgcgccccccgggggaccccacccaagcccccccgccccgagggcacacgaccgggaggcccccagcccaagcagccacgcagccaacgagacaccacccagcacccgagcgcgggcccagcgggggcccagaagcgcaaaaccgagagacgccgccaagaaagccagcccggagcccaccccgcgccgccacggcggcccccccgccaccgggcgaacaaccgccaaaaaagcaaagcaaccaccg

SEQ ID NO: 23 - RNA, 2174 nt, Human
cgggcgaagcccaccgcaggacgggaggagagaaagggaacgaaacaaaaaacgggcacggagaacggaacaaagaagagagggaggagagaaacaagggagggcggggcgggaaagaacgaaaagggagcgcgaagaggagaggcgacagcgcgagccacacgcaagaaaccagaccgccgggagagcggcccagacgcgccaggcgagcaggaggccgcgagggcgggccccagaagccacagggagacggccccccccggccggccggaggaggcgggagcagccccgcgaccacgcgggcggcgcagggccggcggccccggggacggggaggggggcgggaggcacccaaaaccagggggacggcggggggaaccagccgggcaggcgggagagaaagagccggagggagcggccggggcgcagacgccgcagaccacccagacgccggagcccgagccccgacgagccccgcgcccaccgcccgcgccggccgcgccccgccccaccaggccggggccccggcccgcgccgccaccgcgcggcgccgcgcgcgcgggcggcggcgggaccggccacacggccgcgcaggacaacgcacggcccaccaacaagagaccgggcagccccgacggccccggcggccgcgccaggccgcgcgcgggccgggcaggcggcgacgcccacgcgaccccaaggcgcgccaaggcgcgcagagcgcccccaagaacgcccgcacgcgggcggccgaggagcacgcgccgggacaacgaggcccacgaccccgacgcgaccccgagggccgccaaggcgcgccaggcaaccagacgcggggcgggcggaaccgggggcggcgccgcacggacaagggcgaccgagccacgcgcgagagcgggcgcacccaccacacccagaccgcgccaccgccccaccgccggcgcccaaccaccagaccggacgccgagcgaggcggccccgcgagcgcacggcgcaccccaagcgggcggccggcacacgagcagcccaccaccagacgagcgcggcagaacacgccagaaggccgccgggaagggaacggcgagccgccacaccgagagggacacaagggcgagccaccagggccgcggcggccggacgcgcggcgcggagaaccccgcaggggagcgcacgccacaaccggacgagacccccgaagcccagaagcgcccaccgccggcccacgccgcacgggggcgggggccccgcgccggcaggccgccgggacaccaaccggagaaagcggggaagacaagaaggggagacaaggaacgggggaggagaaaggaaccgagcgaggacccggcggggcaggggaggggggggaccggacggacgcccagacccaaggagcacgcccgacccggcgcaaaggagacgacccaaaccgccccccccccgcgcacacaccaggaaagaccggcccagggccccccaccgcgagggaagcacaaaagacccccggccaacaacaggaaaccgacggggcaggaaggaagggaggcacagcgaggaaaaaacaagacgagacaacccgggacggcaagaacgaaccagcgacaggaaggcaaagagagagggaaaagcggacgccgggaaaagagagccacgacaggccacccgaggagaagaggaggaacgggccagagagcccaaccacggaacgaccacaagcgcacgggaaagaagccgcagcgccgaacgcagggagcgaggacagacagcccggaaaccagcaaccagcgcccacgccacgaaggaagcgaaaaggacaaggcgaaaggcgaaaaaaaaaacacgaaaaaaaaagggaaaggcacaaaccagaagcggcaggaaaagcccccagacacacggccaacccaagacgaacaaaaaacaccccccagaagggggcaggcaccggggcccaagaaccaacggccaggaaagcaccaaaaagaaggaaaccccaaacgcaggagggcgagccgagggcacgagaagcaaaacagcgcgagaaaaagaggccagcggcaccaaacaaggcacaccaaggagacagaaaaccagcaagagcgaccccgaacccagcccaccgaaaaagggacaccaagccgagcaaacacaaccaaaggcggggccaagaaaacgac

SEQ ID NO: 24 - RNA, 531 nt, Human
accaaacccaagggaccacacagcccacgcccgaaccagaaaaaaacacagaagcagaaccagcagcagaagcagccgcaccccaccccccagcccagcagcagcagggaaacaaccgccagccccaccccaggaaccagcaccccaaaaccaaggagcccgccaccccaagggccgagcccgccaccccaaaggccgagcccgccagcccaaggccagagcccgccagcccaagggccgagcccgccccaacggcacccagcaccagcccagcagaagaccaagcagaagaagggccacagccagcccgaggagcggccacggaacgaacacccacccacgcagaacccagccagacccgcagagcagcgcacccgaacaaacgcccgcacccaaaaagagcccaccccacggagggcccgagcccgcgaaggcgaacgccacgacgagcagccggccggggcagaggaggaggggaaggacaaggaaccacccagccccaaaaaaacaacccaaaaaaaaaaaaaaaaa

SEQ ID NO: 25 - RNA, 512 nt, Human
accagcaagggaccaacagagacccccacaccaggaccagcacggcagcagagcccagcagcagaagcagccgcaccccaccccccagccagcagcagcagggaaacagccgccagccccacccaggaaccagcaccccaaaaccaaggagcccgccaccccaagggccgagcccgccaccccaaaggcccgagcccgccagcccaaggccagagccagccaccccaagggccgagcccgccccaaagcacccagcaccagcccagcagaagaccaagcagaagaagggccacagccagcccgaggagccggccaccagagcgaaccccacccacgcgagagcccagccgcaaagcacgccccccaaaaaagaaggcagaagccccacacaccgagccgaagaagcgaaggcagaccagagcagcagcgccagaacacgaagagagacaagagaaagcaaagacagccccaacccccaaaacaccaaccaaaaaaaaaaaaaaaaaaaaaaa

SEQ ID NO: 26 - RNA, 537 nt, Human
aaaccccggaccgagcacgacgccggagaaccgaccgagacccagcaggagcacaacagcagcaggcaagcagcccgccagccaccccgggccccacgccaaaggcccagagccagccacccccgaaggcccgagcccgcccaccaccaaaggccacagcccgcccacccagcaggccagcagaaaaccccggacaccccccacccgccagcaaagaccaccgaagagcaagaacagccagaacacaggaccaagaaaggaaaggaaggccacccgccacagcccacccacccacaaagccaccaggaacacagggagcccccagccagaacgcccagagacccgacagcaaaaagccgaggcgccaacgccacgccaggggagacgagcaaaggaagccgggcggccagccccagagccggaagaaagagcagcagccccccgggaaccacagagaacggaggcggcgcgcaccggcacgagccaccaccgcaagcacaccaccccgaaaaagacagcaaaaaaaaaaaaaaaaaa

SEQ ID NO: 27 - RNA, 511 nt, Human
agaggccagcccagacccaaacccggacgagcaccgacgccggagaaccgacgagacccagcaggagcacaacagagcaagaagcagcccgccagccaccccgggccgccgccaaagcgccagagccagccaccccgaaggcccgagcccgcccaccaccaaaggccacagcccgcccacccagcaggccagcagaaaacccgggacaccccccacccgccagccaaaggccacccaagagcaagaacagccggaacacaggaccacgaaaggaagggaaaggccacccgccacagcccaccgcacccacaaagccaccagggaacacagggagccccccagccggacgcccagagaccccgacagcaaaagcccgaggcgccagcgccacgccaggggagacgagcaaaggaagcccagcgaccggcccagagcccggggccacaaagaaagagccgccgcgcaccggcagagccacacggcaagcacccaccccgaaaaagagcagcca

SEQ ID NO: 28 - RNA, 760 nt, Human; identical to SEQ ID NO: 3

(19) United States
(12) Patent Application Publication
(10) Pub. No.: US 2010/0216131 A1
(43) Pub. Date: Aug. 26, 2010

(54) GENE EXPRESSION PROFILING OF ESOPHAGEAL CARCINOMAS

(76) Inventors: Rajyalakshmi Luthra, Missouri City, TX (US); Jaffer A. Ajani, Houston, TX (US); Maden G. Luthra, Missouri City, TX (US)

Correspondence Address: FULBRIGHT & JAWORSKI, LLP, 1301 MCKINNEY, SUITE 5100, HOUSTON, TX, 77010-3095, US

(21) Appl. No.: 12/518,441

(22) PCT Filed: Dec. 10, 2007
(86) PCT No.: PCT/US2007/086948

§ 371 Date: May 14, 2010

Related U.S. Application Data
(60) Provisional application No. 60/874,181, filed on Dec. 11, 2006.

(51) Int. Cl. C12Q 1/00; C07H 21/02

(52) U.S. Cl. 435/6; 536/24.33; 536/23.1

(57) ABSTRACT

The present invention generally regards gene expression profiling of esophageal cancers, including localized esophageal cancers. In particular, gene expression for a particular group of genes identifies individuals that are either going to be responsive to cancer therapy, for example chemotherapy and/or radiation, or that are not going to be responsive to cancer therapy. Exemplary genes having such expression profiles include, for example, PERP, S100A2, and SPRR3.

### FIELD OF THE INVENTION

The present invention is directed at least to the fields of cell biology, molecular biology, cancer biology, and medicine. More particularly, the present invention regards gene expression profiling of esophageal carcinomas, such as localized esophageal carcinomas, for association with pathological response to preoperative chemoradiation, for example. In certain aspects, the invention concerns use of gene expression to determine effectiveness of one or more therapies for esophageal cancer.

### BACKGROUND OF THE INVENTION

Esophageal cancer (ECA) is the ninth most common malignancy in the world and is estimated to be responsible for approximately 13,000 deaths and 14,000 new diagnoses in the United States in 2004 (Jemal et al., 2003; Ilson, 2003). Even when localized, the 5 year survival rate of <20% has not changed significantly in several decades (Ilson, 2003, Enzinger and mayer, 2003). The incidence of adenocarcinomas (ACA) of the esophagus has risen faster than any other malignancy, especially in Caucasian males with an estimated increase in incidence by over 70% in 20 years, thus making ACA the most common histologic type in the West (Pera, 2001; Bolschweiler et al., 2001). Progression of Barrett's metaplasia appears to be one of the major contributors to the observed increase in incidence of ACA (Wild and Hardie, 2003; Winters et al., 1987).

The most common approach to patients with localized carcinoma of the esophagus, irrespective of the histologic type, is preoperative chemoradiotherapy. This approach provides hypothetical advantages including, higher rate of “curative” surgery, reduced local relapse, and early therapy of micrometastases. Due to empiric nature, current approaches lead to considerable uncertainty in patient outcome and result in administration of toxic therapies.

Pre-treatment clinical parameters such as TNM classification, primary location, sex and histologic type are unable to predict differences in the biological behavior of these cancers in patients receiving preoperative chemoradiation (Chirieac et al., 2005). One can, however, predict outcome after surgery by reviewing the AJCC stage. The most favorable survival is noted in patients who do not have any residual cancer in the resected specimen (pathologic complete response or pathCR) (Rohatgi et al., 2005; Berger et al., 2005; Darnton et al., 2003). The fraction of pathCR patients is approximately 25%. However, biomarkers are not available that identify patients who respond to chemoradiotherapy and thus be spared from potentially harmful interventions, and patients who benefit from more aggressive treatments.

Many expression profiling studies have been conducted over the past few years to understand the biology of ECA and to identify biomarkers that can be targeted (Lu et al., 2001; Xu et al., 2002; Xu et al., 2003; Hourihan et al., 2003; Zhou et al., 2003; Ishibashi et al., 2003; Dahlberg et al., 2004; McManus et al., 2004; Luo et al., 2004; Kazemia-Noureini et al., 2004; Brabender et al., 2004). However, these studies lacked treatment and pathologic outcome data to correlate with specific transcriptional signatures. Identification of molecular signatures that predict outcome would be of value in individualizing management of these patients.

### SUMMARY OF THE INVENTION

Esophageal cancer remains one of the most fatal malignancies, with a 5-year survival rate of less than 20%. Over the past two decades, a remarkable shift has occurred in the epidemiology of esophageal cancer, resulting in an alarming increase in the incidence of adenocarcinoma of the proximal esophagus, with a relative decline in the incidence of esophageal squamous cell carcinoma (Pohal, 2005). The incidence of esophageal adenocarcinoma (EAC) is increasing faster than that of any other type of cancer, at a yearly rate of ˜10%, and EAC ranks in the top 15 cancers among Caucasian men in the United States (Pohl, 2005; Pera, 2001; Pera, 2003).

Most EACs arise in the background of Barrett's esophagus, which is characterized by replacement of normal squamous epithelium with metaplastic columnar epithelium owing to chronic reflux esophagitis (Spechler, 2002). When EAC is local-regional, preoperative chemoradiation (CTXRT) is commonly recommended. However, the role of preoperative CTXRT remains controversial. When CTXRT is used, longer survival is noted in a small fraction (about 27%) of patients who achieve pathologic complete response (pathCR) (Chirieac et al., 2005; Rohatgi et al., 2005; Berger et al., 2005; Darnton et al., 2003; Rohatgi et al., 2005); however, patients who are likely to have a pathCR cannot be predicted by the pretreatment clinical parameters. Thus, it is of paramount importance to discover biomarkers that can predict response to CTXRT in order to individualize and optimize therapy for this group of patients. In specific aspects, individualization of therapy is employed through studying the molecular biology of EAC and the patient's genetics.

The present invention concerns profiles of exemplary pre-treatment endoscopic cancer biopsies from patients, for example with ECA using Affymetrix U133A (Santa Clara, Calif.) and correlation of their exemplary molecular profiles with pathologic response. The expression levels of a few genes selected based on array data were assessed by polymerase chain reaction (PCR), for example, as biomarkers of pathologic response. In addition, the inventors identified from the microarray data (such as by using Ingenuity Pathway Analysis Software (Mountain View, Calif.), for example) to identify key biologic pathways and functions associated with chemoradiation resistance.

In specific embodiments, there was substantially lower expression of at least the S100A2 (S100 calcium binding protein A2) and SPRR3 (small proline-rich protein 3 or esophagin) genes in pretreatment cancer specimens of patients resistant to CTXRT (Luthra et al., 2006). Loss of expression of both S100A2 and SPRR3 has been associated with premalignant and malignant states, including cancers of the lung, esophagus, and cervix (Nagy et al., 2001; Hitomi et al., 1998; Hibi et al., 2003; Chen et al., 2000; Smolinski et al., 2002; Kimos et al., 2004; Suzuki et al., 2005; Shimada et al., 2005). Both genes are located in an evolutionarily conserved genetic cluster, designated as the epidermal differentiation complex (EDC), at the 1q21 chromosomal band (Volz et al., 1993; Mischke et al., 1996; Steinert et al., 1998). This genetic region encompassing 2 Mb of genomic DNA harbors three gene families and approximately 43 genes that are involved in terminal squamous differentiation of the human epidermis (Volz et al., 1993). A recent study has suggested that the expressional down-regulation of some EDC complex genes, including S100A2 and SPRR3, may serve as a potential marker of progression to EAC (Kimchi et al., 2005).

In additional specific aspects of the invention, the EDC gene cluster (see FIG. 7 for an exemplary schematic of the epidermal differentiation complex at 1q21) is differentially expressed in CTXRT-sensitive and -resistant EAC, and in further specific embodiments, this reflects distinct biologic EAC entities. To characterize this embodiment, pretreatment specimens of EAC and normal squamous esophageal mucosa were compared for the expression of approximately 517 genes in the 1q21-q25 chromosomal region that included the EDC cluster and its flanking regions and selected target genes based on their maximal differential expression. Then, using pathCR after therapy as an endpoint, the association between the expression levels of 1q21-1q25 target genes and response to CTXRT and clinical outcome were analyzed.

In some aspects of the invention, gene expression profiling in accordance with the invention determines whether or not an individual is resistant to one or more cancer therapies, will become resistant to one or more cancer therapies, or is susceptible to becoming resistant to one or more cancer therapies. In other embodiments, gene expression profiling in accordance with the invention determines whether or not an individual has cancer or is at risk for having cancer. In particular embodiments, identification of biomarkers for targeted therapy occurs by comparison of gene expression profiles of normal squamous mucosa (NSM) with adenocarcinoma (ACA). In certain aspects, the gene expression profile utilizes software to identify key functions and pathways that distinguish different biological states (such as Ingenuity Pathway Analysis Software, for example). Such software may utilize a relational database with many individually modeled relationships between proteins, genes, cells, tissues, drugs, and diseases, for example.

In particular aspects of the invention, differential gene expression related to determining whether or not an individual will have complete or less than complete pathologic response will be related to one or more of the same pathways. For example, one or more members of the same pathway may be differentially expressed between pCR responders and <pCR responders. Although any pathway may be involved in the differential expression associated with esophageal cancer, in specific embodiments the pathway concerns NF-κB signaling (which may be upregulated, for example, and be associated with poor response to chemoradiation therapy). Therefore, genes in the NF-κB pathway serve as targets for treatment for esophageal cancer, in particular embodiments.

Although the invention is particularly suited for esophageal cancer, in alternative embodiments, one or more of the genes of the invention are useful for gene expression profiling of brain cancer, lung cancer, prostate cancer, bladder cancer, breast cancer, liver cancer, colon cancer, skin cancer, kidney cancer, pancreatic cancer, ovarian cancer, testicular cancer, esophageal cancer, cervical cancer, gall bladder cancer, lymphoma, leukemia, thyroid cancer, or salivary gland cancer, for example.

Gene expression for the invention may be identified by any suitable means, although in specific aspects it is identified through nucleic acid or protein (or both), such as through nucleic acid levels or protein levels, or both. Nucleic acid may be assayed by any suitable method, although in specific embodiments it is assayed by polymerase chain reaction (for example by realtime polymerase chain reaction), expressed sequence tag (EST) sequencing, cDNA microarray hybridization, subtractive cloning, differential display, MARD (Cheng et al., 2006) and serial analysis of gene expression (SAGE). Protein may be assayed by any suitable method, although in specific embodiments it is assayed using an antibody, for example by immunohistochemistry or western.

In certain embodiments of the invention, the expression levels of one or more of SEQ ID NO:1, SEQ ID NO:2, SEQ ID NO:3, SEQ ID NO:4, SEQ ID NO:5, or SEQ ID NO:6 are assayed to determine whether or not an individual will have pathologic complete response to chemotherapy, radiation, or both, for esophageal cancer. In certain embodiments of the invention, the expression levels of one or more of SEQ ID NO:1, SEQ ID NO:2, SEQ ID NO:3, SEQ ID NO:4, SEQ ID NO:5, or SEQ ID NO:6 are assayed to determine whether or not an individual will respond to one or more cancer therapies, including chemotherapy, radiation, or both. Additional genes for which expression level may be determined include, for example, annexin 1 (GenBank® Accession No. BC018683; SEQ ID NO:7), SPRRS, S100A8 (GenBank® Accession No. NM_{—}002964; SEQ ID NO:8), A9, TGM3 (GenBank® Accession No. NM_{—}003245; SEQ ID NO:9), CK4 (GenBank® Accession No. X58328; SEQ ID NO:10), CK13, SK15, Shh (GenBank® Accession No. L38518; SEQ ID NO:11), Gli-1 (GenBank® Accession No. NM_{—}005269; SEQ ID NO:12), MMP9 (GenBank® Accession No. NM_{—}004994; SEQ ID NO:13), FOXO3, TRAF2 (GenBank® Accession No. BC064662; SEQ ID NO:14), Bmi1 (GenBank® Accession No. AY011539; SEQ ID NO:15, Sox2 (GenBank® Accession No. NM_{—}003106; SEQ ID NO:16), and ATP binding cassette subfamily C.

In particular aspects of the invention, the level of expression of one or more genes is determined in a sample from an individual that has cancer, is suspected of having cancer, or that will be receiving chemotherapy, radiation, or both, for example. In specific embodiments, the level of expression is relative between expression of the same gene from two different sources, including between one gene from a sample from the individual and the same gene from another source. In specific embodiments, the level of expression is relative to the level of expression in a control, such as a control provided in a commercial kit, including one from a normal sample; a control obtained from one or more non-cancerous cells from the same individual or another individual, including from the same organ or tissue; or a control obtained from non-cancerous cells from the same organ, for example. Expression levels may compared to a gene that does not change substantially among an individual population for a particular cancer, such as 18S RNA, GAPDA, and so forth. Expression levels may be compared to another esophageal cancer sample, such as a tumor sample, from another individual. The differential relative expression of the same gene from two samples or a sample and a control may be, for example, about two-fold or greater, including about three-fold, about four-fold, about five-fold, about six-fold, about seven-fold, about eight-fold, about nine-fold, about ten-fold, about fifteen-fold, about 20-fold, about 25-fold, about 30-fold, about 40-fold, about 50-fold, about 60-fold, about 70-fold, about 80-fold, about 90-fold, or about 100-fold, about 125-fold, about 150-fold, about 175-fold, about 200-fold, or more.

In particular embodiments, the expression levels of two or more genes differentially identify whether or not an individual will respond to one or more cancer therapies. For example, an individual may be identified as being non-responsive to a therapy because one gene expression level is X-fold relative gene expression and because another gene expression level is Y-fold relative gene expression, wherein X and Y are nonidentical numbers.

Thus, in one embodiment of the invention there is a method of determining effectiveness of a cancer therapy in an individual with cancer, comprising assaying gene expression levels in a sample from the individual, said sample comprising RNA or protein, wherein said assaying comprises determining the expression, such as the expression level, of one or more genes of the epidermal differentiation complex (EDC) of chromosome 1q21-25. In a specific embodiment, the method is further defined as discriminating whether or not the individual will have pathologic complete response or less than pathologic complete response. In specific embodiments, assaying comprises determining the expression, such as the level of expression, of two or more, three or more, or four or more genes of the EDC.

In an additional embodiment, there is a method of determining effectiveness of a cancer therapy in an individual with cancer, comprising assaying gene expression levels in a sample from the individual, said sample comprising RNA or protein, wherein said assaying comprises determining the expression, such as the expression level, of one or more polynucleotides selected from the group consisting of SEQ ID NO:1, SEQ ID NO:2, SEQ ID NO:3, SEQ ID NO:4, SEQ ID NO:5, and SEQ ID NO:6. In specific embodiments, assaying comprises subjecting a substrate having nucleic acids to RNA from the sample. In other specific embodiments, assaying comprises subjecting cDNA from the RNA from the sample to polymerase chain reaction. In additional aspects, assaying comprises using antibodies to assay for proteins from the sample. In particular embodiments, the cancer is esophageal, and in further specific embodiments, the cancer therapy comprises chemotherapy, radiation, surgery, or a combination thereof.

In some embodiments of the invention, methods further comprise obtaining a sample from the individual, such as by biopsy, obtaining saliva, obtaining gastric juice, or a combination thereof. The sample may be obtained by a health care provider or by another.

In some aspects of methods that employ a substrate having nucleic acids affixed thereto, there may be one or more nucleic acids affixed to the substrate that anneal under stringent conditions to at least two, three, four, five, or all of the sequences in the RNA of a sample, wherein the sequences are selected from the group consisting of SEQ ID NO:1, SEQ ID NO:2, SEQ ID NO:3, SEQ ID NO:4, SEQ ID NO:5, and SEQ ID NO:6.

In additional aspects of the invention that employ substrates having nucleic acids affixed thereto, at least about 10%, 20%, 30%, 40%, 50%, 60%, 70%, 80%, 90%, or 95%, of the nucleic acids on the substrate are capable of hybridizing under stringent conditions to RNA in a sample. In additional aspects of the invention that employ substrates having nucleic acids affixed thereto, substantially all of the nucleic acids on the substrate are capable of hybridizing under stringent conditions to RNA in a sample.

In specific embodiments, methods of the invention may further comprise subjecting the individual to x-ray, barium swallow, biopsy, esophagoscopy, or a combination thereof. In other aspects, when the effectiveness of the cancer therapy is determined to be non-effective, the individual is provided with an alternative therapy, such as one that comprises surgery, chemotherapy, radiation, or a combination thereof, for example. In some aspects of the invention, a decrease in expression level of one or more particular genes from a sample from the individual indicates that the individual will be responsive to one or more certain therapies. In other aspects of the invention, an increase in expression level of one or more particular genes from a sample from the individual indicates that the individual will be responsive to one or more certain therapies. In still other aspects of the invention, there is an increase in expression level of one or more particular genes from a sample from the individual and there is a decrease in expression level of one or more particular other genes from a sample from the individual to indicate whether the individual will be responsive to one or more certain therapies.

In one embodiment of the invention, there is an isolated substrate, comprising two or more of polynucleotides affixed thereto, wherein said polynucleotides are selected from the group consisting of SEQ ID NO:1, SEQ ID NO:2, SEQ ID NO:3, SEQ ID NO:4, SEQ ID NO:5, and SEQ ID NO:6. In other aspects of the invention, there is a plurality of primers suitable for use in amplifying one or more of the polynucleotides are selected from the group consisting of SEQ ID NO:1, SEQ ID NO:2, SEQ ID NO:3, SEQ ID NO:4, SEQ ID NO:5, and SEQ ID NO:6.

In particular embodiments of the invention, there is a kit for determining effectiveness of a cancer therapy, said kit housed in a suitable container, comprising a substrate having one or more of SEQ ID NO:1, SEQ ID NO:2, SEQ ID NO:3, SEQ ID NO:4, SEQ ID NO:5, and SEQ ID NO:6 attached thereto or antibodies that react immunologically to SEQ ID NO:1, SEQ ID NO:2, SEQ ID NO:3, SEQ ID NO:4, SEQ ID NO:5, or SEQ ID NO:6. In some cases, methods of the invention further comprises one or more sample-obtaining apparatuses, such as a swab, toothpick, scalpel, spatula, or syringe, for example. In certain aspects, the kit further comprises one or more chemotherapeutic drugs, such as 5-fluorouracil (5-FU), cisplatin, carboplatin, bleomycin, mitomycin, doxorubicin, methotrexate, paclitaxel, vinorelbine, topotecan, irinotecan, or a combination or mixture thereof. In additional embodiments, the substrate comprises microchip, microplate, column, or filter.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows cluster analysis showing molecular subtypes of esophageal cancers. Five of the six cases with pathCR were clustered in Type I. R indicates cancers with pathCR in resected esophagogastrectomy specimens.

FIG. 2 provides an exemplary network profile generated by Ingenuity Pathway Analysis highlights downregulation of biomarkers associated with apoptotic and proliferative pathways in esophageal cancer type II in comparison to type I. The networks are displayed graphically as nodes (genes/gene products) and edges (the biological relationships between the nodes). The intensity of the node color indicates the degree of up- (green) or down-(red) regulation.

FIG. 3 shows real time qPCR expression levels of [symbol] ( ), [symbol] ( ) and [symbol] ( ). Relative expression value for each gene was calculated as described in Methods using 2^{-Δ CT} method. R indicates cancers that had pathCR (R). Red lines with dots and dashes indicate expression cut off values of 10 and 100 respectively. 9/17 cancers (8/9 from subtype I and 1/8 from subtype II) showed expression values>10 in at least two of the three markers. Only 7/17 cancers (all from subtype I) had expression values>100 in at least two of the three marker genes. These seven included five cancers that achieved pathCR (cancers 1, 3, 16, 56, and 23).

FIG. 4 shows treeview displays of unsupervised cluster analysis of esophageal cancers and normal squamous mucosa (NSM). Expression data of 517 genes from the chromosome 1q21-25 region were used for cluster analysis as described in Methods. All NSMs clustered together and segregated from cancers (4A and 4B). However, cancers segregated into two clusters designated as subgroups I and II (4A and 4B). Four of the five cancers that achieved pathologic complete response (pathCR), denoted by red stars, clustered in subgroup I. FIG. 4C shows the genes with marked difference in expression between the subgroups.

FIG. 5 provides average expression levels of genes differentially expressed in normal and cancer specimens. Graph highlights the dramatic drop in transcription of genes within and in close proximity to the epidermal differentiation complex cluster in esophageal cancers, compared with normal squamous mucosa (NSM), and divergence of the two molecular subgroups based on the expression levels of these genes. All genes showing equal or greater than twofold difference in expression were plotted in the order that matched their location on the chromosome, from centromere to telomere.

FIG. 6 shows real-time qPCR expression levels of 9 exemplary genes, ECM1, CRNN, NICE-1, IVL, SPRR3, S100A2, ADAR, CRABP2, and RGS5. FIG. 6A is a schematic of the chromosome 1 with a detailed map of the epidermal differentiation complex and flanking genes. The genes shown in bold were analyzed by qPCR (B). Relative expression values of the selected genes shown in FIG. 6B were calculated as described in Example 3 using the 2^{-ΔC}_{T} method. The relative gene expression values for pooled normal squamous mucosal specimens (NSM) are shown in blue. The relative expression levels in cancers from subgroups I and II are shown in red and green, respectively. The numbers shown in the legend key correspond to patient numbers in Tablet. The dotted lines show the expression levels used for dichotomization into high/low expression (high expression was defined as relative expression levels of >100 for CRNN and >5000 for NICE-1 and IVL).

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein.

### I. General Embodiments of the Invention

Individuals with localized esophageal carcinoma have a 5-yr survival rate of <20%. Individuals are often treated similarly (i.e. preoperative chemoradiation), but the outcomes vary greatly. Chemoradiation and surgery can result in significant undesirable consequences. Currently, however, there are no tools to help select optimum therapy. In certain aspects, the invention concerns gene expression profiling to identify biomarkers of resistance to therapy. Pretreatment endoscopic cancer biopsies from 19 patients (16 with adenocarcinoma, 2 with squamous cell carcinoma and 1 with adenosquamous carcinoma) enrolled in a uniform preoperative chemoradiation protocol were profiled using a microchip. Surgical speciments following therapy were assessed for the degree of pathologic response. Based on array data, selected genes were analyzed by polymerase chain reaction. Unsupervised hierarchical cluster analysis segregated the cancers into two molecular subtypes, each consisting of 10 and 9 speciments, respectively. Most cancers (5/6) that had pathologic complete response (pathCR) clustered in molecular subtype I. Subtype II, with one exception, consisted of cancers that had less than pathCR (<pathCR). Using a combination marker approach, levels of multiple genes were able to discriminate pathCR from <pathCR. In specific embodiments, the levels of at least three genes, PERP (a novel effector involved in p53-dependent apoptosis), S100A2 (a calcium binding protein; in specific embodiments it may be a tumor suppressor gene), and SPRR3 (a member of small proline-rich proteins; also known as esophagin; is a component of the epithelial cell envelope) allowed discrimination of pathCR from <pathCR with high sensitivity and specificity (85%). Ingenuity pathway (Ingenuity Systems; Redwood City, Calif.) analysis identified apoptotic pathway as one of the key functions downregulated in molecular type II in comparison with type I. Thus, in particular embodiments, expression profiling distinguishes cancer with different pathologic outcome. In general aspects, the invention relates to subtypes of esophageal cancers with distinct molecular signatures. In other aspects, the invention provides biomarkers of resistance to pre-operative chemoradiation in patients with esophageal cancer. In other embodiments, expression levels of the identified biomarkers in pre-treatment cancer biopsies, for example, provide a means of identifying individuals that are likely to respond and thus can be spared from harmful interventions and individuals that are resistant to therapy and thus require alternate therapies. The invention also provides targetable pathways for diagnosis and/or treatment of cancer, in specific aspects. Compositions of the invention include, for example, panels of markers for therapy resistance that can be analyzed by any suitable method in the art, for example using an Affymetrix® chip, realtime polymerase chain reaction, western and/or immunohistochemistry.

### II. Esophageal Cancer

The esophagus is the hollow, muscular tube that allows passage of food and liquid from the throat to the stomach. Esophageal walls are made up of more than one layer of tissue, including mucous membrane, muscle, and connective tissue. Esophageal cancer begins at the inside lining of the esophagus and spreads outward through the other layers as it develops. Squamous cell carcinoma and adenocarcinoma are the two most common forms of esophageal cancer. In squamous cell carcinoma, cancer forms in squamous cells, which are flat, thinly shaped cells that line the esophagus. It is most likely to develop in the upper and middle part of the esophagus, but it can occur at any point along the esophagus. Adenocarcinoma is a cancer that begins in glandular (secretory) cells, which line the esophagus to produce and release fluids, for example mucus. These types of esophageal cancers are more often found in the lower part of the esophagus, near the stomach. The present invention is useful at least for any type of esophageal cancer.

Certain factors that put an individual at risk for developing esophageal cancer include one or more of the following: tobacco use; heavy alcohol use; Barrett's esophagus (a condition where cells lining the lower part of the esophagus have changed or been replaced with abnormal cells); gastric reflux; older age; male, and being African-American. Signs of esophageal cancer include one or more of the following: painful swallowing; difficult swallowing; weight loss; pain; hoarseness; cough; indigestion; and heartburn. Exemplary present methods for diagnosing esophageal cancer include chest x-ray, barium swallow, esophagoscopy, biopsy, or a combination thereof. In particular aspects, the methods of gene expression profiling are used alternative to or in addition to one of these exemplary diagnosing methods. In certain aspects, an individual is identified as being at risk for developing esophageal cancer and/or has one or more signs of esophageal cancer.

Primary treatment modalities include surgery (esophagogastrectomy or esophagectomy, for example), chemotherapy, photodynamic therapy, and/or radiation therapy, for example. Combined modality therapy (i.e., chemotherapy plus surgery, or chemotherapy and radiation therapy plus surgery) may be employed. Standard chemotherapy drugs for esophageal cancer include at least one or more of the following: 5-fluorouracil (5-FU), cisplatin, carboplatin, bleomycin, mitomycin, doxorubicin, methotrexate, paclitaxel, vinorelbine, topotecan, and irinotecan. In chemoradiotherapy, the most frequently used drugs are 5-FU and cisplatin administered concomitantly, in specific embodiments.

### III. Nucleic Acid Compositions

In certain embodiments of the present invention, particular sequences are employed in the inventive gene expression profiling methods and compositions. Although a skilled artisan recognizes that these specific sequences may be employed exactly as provided herein, in other embodiments sequences that are similar to those exemplary sequences provided herein are also useful.

In specific embodiments, one or more polynucleotides of the invention are present on a substrate for assaying gene expression, for example. Exemplary sequences and their National Center for Biotechnology Information GenBank's database Accession Number include PERP (NM_{—}022121; SEQ ID NO:1); S100A2 (NM_{—}005978; SEQ ID NO:2); SPRR3 (NM_{—}005416; SEQ ID NO:3); IVL (NM_{—}005547; SEQ ID NO:4); CRNN (NM_{—}016190; SEQ ID NO:5); NICE-1 (AJ243662; SEQ ID NO:6). Such sequences or fragments thereof may be employed at least in part on substrates for gene expression profiling for esophageal cancer. Such fragments must be at least long enough to distinguish themselves from being from other genes.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

A. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, carboxyalkyl, amino, hydroxyl, halogen (i.e., fluoro, chloro, bromo, or iodo), thiol or alkylthiol moeity. Preferred alkyl (e.g., alkyl, carboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleoside or nucleotide, using any chemical or natural synthesis method described herein or known to one of ordinary skill in the art.

B. Nucleosides

As used herein, a “nucleoside” refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9 position of a purine or a 7-deazapurine to the 1′-position of a 5-carbon sugar. In another non-limiting example, a nucleoside comprising a pyrimidine nucleobase (i.e., C, T or U) typically covalently attaches a 1 position of a pyrimidine to a 1′-position of a 5-carbon sugar (Kornberg and Baker, 1992).

C. Nucleotides

As used herein, a “nucleotide” refers to a nucleoside further comprising a “backbone moiety”. A backbone moiety generally covalently attaches a nucleotide to another molecule comprising a nucleotide, or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. However, other types of attachments are known in the art, particularly when a nucleotide comprises derivatives or analogs of a naturally occurring 5-carbon sugar or phosphorus moiety.

D. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference).

Additional non-limiting examples of nucleosides, nucleotides or nucleic acids comprising 5-carbon sugar and/or backbone moiety derivatives or analogs, include those in U.S. Pat. No. 5,681,947 which describes oligonucleotides comprising purine derivatives that form triple helixes with and/or prevent expression of dsDNA; U.S. Pat. Nos. 5,652,099 and 5,763,167 which describe nucleic acids incorporating fluorescent analogs of nucleosides found in DNA or RNA, particularly for use as fluorescent nucleic acids probes; U.S. Pat. No. 5,614,617 which describes oligonucleotide analogs with substitutions on pyrimidine rings that possess enhanced nuclease stability; U.S. Pat. Nos. 5,670,663, 5,872,232 and 5,859,221 which describe oligonucleotide analogs with modified 5-carbon sugars (i.e., modified 2′-deoxyfuranosyl moieties) used in nucleic acid detection; U.S. Pat. No. 5,446,137 which describes oligonucleotides comprising at least one 5-carbon sugar moiety substituted at the 4′ position with a substituent other than hydrogen that can be used in hybridization assays; U.S. Pat. No. 5,886,165 which describes oligonucleotides with both deoxyribonucleotides with 3′-5′ internucleotide linkages and ribonucleotides with 2′-5′ internucleotide linkages; U.S. Pat. No. 5,714,606 which describes a modified internucleotide linkage wherein a 3′-position oxygen of the internucleotide linkage is replaced by a carbon to enhance the nuclease resistance of nucleic acids; U.S. Pat. No. 5,672,697 which describes oligonucleotides containing one or more 5′ methylene phosphonate internucleotide linkages that enhance nuclease resistance; U.S. Pat. Nos. 5,466,786 and 5,792,847 which describe the linkage of a substituent moeity which may comprise a drug or label to the 2′ carbon of an oligonucleotide to provide enhanced nuclease stability and ability to deliver drugs or detection moieties; U.S. Pat. No. 5,223,618 which describes oligonucleotide analogs with a 2 or 3 carbon backbone linkage attaching the 4′ position and 3′ position of adjacent 5-carbon sugar moiety to enhanced cellular uptake, resistance to nucleases and hybridization to target RNA; U.S. Pat. No. 5,470,967 which describes oligonucleotides comprising at least one sulfamate or sulfamide internucleotide linkage that are useful as nucleic acid hybridization probe; U.S. Pat. Nos. 5,378,825, 5,777,092, 5,623,070, 5,610,289 and 5,602,240 which describe oligonucleotides with three or four atom linker moeity replacing phosphodiester backbone moeity used for improved nuclease resistance, cellular uptake and regulating RNA expression; U.S. Pat. No. 5,858,988 which describes hydrophobic carrier agent attached to the 2′-O position of oligonucleotides to enhanced their membrane permeability and stability; U.S. Pat. No. 5,214,136 which describes oligonucleotides conjugated to anthraquinone at the 5′ terminus that possess enhanced hybridization to DNA or RNA; enhanced stability to nucleases; U.S. Pat. No. 5,700,922 which describes PNA-DNA-PNA chimeras wherein the DNA comprises 2′-deoxy-erythro-pentofuranosyl nucleotides for enhanced nuclease resistance, binding affinity, and ability to activate RNase H; and U.S. Pat. No. 5,708,154 which describes RNA linked to a DNA to form a DNA-RNA hybrid.

E. Preparation of Nucleic Acids

A nucleic acid may be made by any technique known to one of ordinary skill in the art, such as for example, chemical synthesis, enzymatic production or biological production. Non-limiting examples of a synthetic nucleic acid (e.g., a synthetic oligonucleotide), include a nucleic acid made by in vitro chemically synthesis using phosphotriester, phosphite or phosphoramidite chemistry and solid phase techniques such as described in EP 266,032, incorporated herein by reference, or via deoxynucleoside H-phosphonate intermediates as described by Froehler et al., 1986 and U.S. Pat. No. 5,705,629, each incorporated herein by reference. In the methods of the present invention, one or more oligonucleotide may be used. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

A non-limiting example of an enzymatically produced nucleic acid include one produced by enzymes in amplification reactions such as PCR™ (see for example, U.S. Pat. No. 4,683,202 and U.S. Pat. No. 4,682,195, each incorporated herein by reference), or the synthesis of an oligonucleotide described in U.S. Pat. No. 5,645,897, incorporated herein by reference. A non-limiting example of a biologically produced nucleic acid includes a recombinant nucleic acid produced (i.e., replicated) in a living cell, such as a recombinant DNA vector replicated in bacteria (see for example, Sambrook et al. 1989, incorporated herein by reference).

F. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

G. Nucleic Acid Segments

In certain embodiments, the nucleic acid is a nucleic acid segment. As used herein, the term “nucleic acid segment,” are smaller fragments of a nucleic acid, such as for non-limiting example, those that comprise only part of the regulatory sequences for a given transcribed polynucleotide.

H. Nucleic Acid Complements

The present invention also encompasses a nucleic acid that is complementary to a nucleic acid of the invention. In particular embodiments the invention encompasses a nucleic acid or a nucleic acid segment complementary to the sequence set forth in SEQ ID NO:1, SEQ ID NO:2, SEQ ID NO:3, SEQ ID NO:4, SEQ ID NO:5, and SEQ ID NO:6, for example. A nucleic acid is “complement(s)” or is “complementary” to another nucleic acid when it is capable of base-pairing with another nucleic acid according to the standard Watson-Crick, Hoogsteen or reverse Hoogsteen binding complementarity rules. As used herein “another nucleic acid” may refer to a separate molecule or a spatial separated sequence of the same molecule.

As used herein, the term “complementary” or “complement(s)” also refers to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, to about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In certain embodiments, a “partly complementary” nucleic acid comprises a sequence that may hybridize in low stringency conditions to a single or double stranded nucleic acid, or contains a sequence in which less than about 70% of the nucleobase sequence is capable of base-pairing with a single or double stranded nucleic acid molecule during hybridization.

I. Hybridization

As used herein, “hybridization”, “hybridizes” or “capable of hybridizing” is understood to mean the forming of a double or triple stranded molecule or a molecule with partial double or triple stranded nature. The term “anneal” as used herein is synonymous with “hybridize.” The term “hybridization”, “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions”, and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suite a particular application.

The nucleic acid(s) of the present invention, regardless of the length of the sequence itself, may be combined with other nucleic acid sequences, including but not limited to, promoters, enhancers, polyadenylation signals, restriction enzyme sites, multiple cloning sites, coding segments, and the like, to create one or more nucleic acid construct(s). As used herein, a “nucleic acid construct” is a nucleic acid engineered or altered by the hand of man, and generally comprises one or more nucleic acid sequences organized by the hand of man.

In a non-limiting example, one or more nucleic acid constructs may be prepared that include a contiguous stretch of nucleotides identical to or complementary to promoter sequences of the invention, for example. A nucleic acid construct may be about 3, about 5, about 8, about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

The exemplary term “a sequence essentially as set forth in SEQ ID NO:4”, for example, means that the sequence substantially corresponds to a portion of SEQ ID NO:4 and has relatively few nucleotides that are not identical to, or a biologically functional equivalent of, the nucleotides of SEQ ID NO:4. Thus, “a sequence essentially as set forth in SEQ ID NO:4” encompasses nucleic acids, nucleic acid segments, and genes that comprise part or all of the nucleic acid sequences as set forth in SEQ ID NO:4. SEQ ID NO:4 is referred to herein solely as an illustrative embodiment, and one of skill in the art recognizes that such description analogously applies to other specific sequences of the invention.

The term “biologically functional equivalent” is well understood in the art and is further defined in detail herein. Accordingly, a sequence that has between about 70% and about 80%; or more preferably, between about 81% and about 90%; or even more preferably, between about 91% and about 99%; of nucleotides that are identical or functionally equivalent to the nucleotides of sequences referred to herein, such as the exemplary SEQ ID NO:1, SEQ ID NO:2, SEQ ID NO:3, SEQ ID NO:4, SEQ ID NO:5, or SEQ ID NO:6 will be a sequence that is respectively “essentially as set forth in the SEQ ID NO:1, SEQ ID NO:2, SEQ ID NO:3, SEQ ID NO:4, SEQ ID NO:5, or SEQ ID NO:6”, provided the biological activity of the sequences is maintained.

In certain other embodiments, the invention concerns at least one recombinant vector that include within its sequence a nucleic acid sequence essentially as set forth in SEQ ID NO:1, SEQ ID NO:2, SEQ ID NO:3, SEQ ID NO:4, SEQ ID NO:5, or SEQ ID NO:6.

### IV. Combination Diagnostics

In certain embodiments of the invention, the present inventive gene expression profiling is used in conjunction with one or more diagnostic or prognostic methods in the art. Exemplary present methods for diagnosing and/or prognosticating esophageal cancer include chest x-ray, barium swallow, esophagoscopy, biopsy, or a combination thereof, so in specific aspects of the invention a sample from an individual is subjected to the gene expression profiling of the present invention in addition to other diagnostic methods or compositions. Such a combination may provide information whether or not an individual will have cancer, whether or not an individual is high risk for developing cancer, or whether or not an individual will become resistant to one or more cancer therapies.

The present invention may occur concomitantly with another diagnosing or prognosticating method or it may occur at different times, such as before and/or after one of the other therapies. For example, one may utilize the gene expression profiling of the present invention prior to radiation and/or chemotherapy to determine whether or not the radiation and/or chemotherapy will be effective in the individual, such as whether or not the radiation and/or chemotherapy will become resistant in the individual. In other embodiments, the present invention is applied to an individual following onset of chemotherapy treatment to determine whether or not the cancer is responding to the treatment, for example.

### V. Kits of the Invention

Any of the compositions described herein may be comprised in a kit. In a non-limiting example, the kit comprises a composition suitable for gene expression profiling for esophageal cancer. Exemplary embodiments include antibodies and/or a substrate that comprises nucleic acids. For example, the substrate may have particular nucleic acids attached thereto. In particular aspects, the substrate comprises RNA, DNA, or both attached thereto. In other aspects, the substrate comprises one or more nucleic acids described herein. The substrate may be of any suitable kind so long as gene expression is able to be determined, but in specific embodiments, the substrate is a microchip, microarray, filter, plate, and so forth.

In other embodiments of the invention, the kit comprises one or more apparatuses to obtain a sample from an individual. Such an apparatus may be one or more of a swab, such as a cotton swab, toothpick, scalpel, spatula, syringe, and so forth, for example.

In additional embodiments, the kit further comprises one or more cancer therapies, such as would be used alternatively to the therapy for which the invention determined the individual was resistant thereto. In specific but exemplary embodiments, the alternative cancer therapy comprises one or more of 5-fluorouracil (5-FU), cisplatin, carboplatin, bleomycin, mitomycin, doxorubicin, methotrexate, paclitaxel, vinorelbine, topotecan, and irinotecan. If such compositions are provided in the kits, they may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods for Example 2

The present invention concerns exemplary materials and methods that may be used in at least certain embodiments of the invention.

### Patient Selection and Evaluation

All patients in this report participated in a clinical trial approved by the Institutional Review Board. Patients with localized histologically confirmed squamous cell carcinoma (SCCA) or ACA of the thoracic esophagus were considered eligible. Patients were evaluated by chest radiograph, computerized tomography of the chest and abdomen, upper gastrointestinal double-contrast barium radiographs, an esophago-gastro-duodenoscopy with endoscopic ultrasonography (EUS), electrocardiogram, SMA-12, electrolytes, complete blood count including platelet count, and serum baseline carcinoembryonic antigen (CEA) level. Positron emission tomography (PET) was performed when available. Patients with T2-3 with any N, patients with M1a cancer (celiac nodes associated with a GEJ carcinoma) and patients with T1N1 carcinoma were considered eligible. All patients were evaluated prior to registration by a multidisciplinary team that included thoracic oncology surgeons, radiation oncologists, gastroenterologists, and medical oncologists. Eligible patients had to have cancer that was considered technically resectable and medically operable based on the clinical staging and evaluation. All patients signed a written informed consent, which was approved by the Institutional Review Board.

Patients with T4 cancer and patients with T1N0 lesions were excluded. Patients with any evidence of metastatic cancer were also not enrolled. Patients with uncontrolled medical conditions (such as diabetes, hypertension, heart condition classified as NYHA class III or IV, or psychiatric illness) were not eligible. Patients who could not comprehend the purpose of this clinical trial or comply with its requirements were not enrolled.

### Treatment

The objective of the protocol was to determine the feasibility of the 3-step approach using 3 chemotherapy agents prior to and during preoperative chemoradiation. If a patient had an R0 resection, no further therapy was planned. Patients who underwent an R1 resection (microscopic carcinoma at the margin) or R2 resection (gross carcinoma after surgery) or who had M1 disease were offered palliative care.

Step 1: Induction Chemotherapy

All patients had a central venous line placed prior to starting chemotherapy. Patients received docetaxel as i.v. bolus (at 33 mg/m²), irinotecan (at 55 mg/m²) as i.v. bolus, and 5-fluorouracil (at 2 g/m²) infusion over 24 hours weekly for 2 weeks followed by 1 week off. One cycle was 6-week long. If there was no cancer progression after the first cycle, patients received a second cycle of the induction chemotherapy. Standard premedications were used.

Step 2: Preoperative Chemoradiotherapy

Patients received up to 50.4 Gy of radiotherapy in 28 fractions. Concurrently, patients received docetaxel (20 mg/m²) i.v. bolus weekly, irinotecan (30 mg/m²) i.v. bolus weekly, and 5-fluorouracil (300 mg/m²/24 hours as continuous infusion Monday through Friday of each radiotherapy week). Standard premedications were used.

Step 3: Surgery

Approximately 5 to 6 weeks following the end of chemoradiotherapy, patients were restaged fully. If there was no contraindication for surgery, patients underwent an attempted surgical resection of the primary and regional nodes. Patients were then followed for 5 years or until death.

### Tissue Collection

Patients undergoing therapeutic or diagnostic endoscopic procedure volunteered in an approved tissue collection protocol, thus allowing collection and storage of specimens (blood and cancer tissue). Up to 15 biopsy specimens (8 cancer, 4 junction and 3 normal tissues) were collected. The size of usual biopsy was approximately 1.0 mm and tissue specimens were snap frozen in liquid nitrogen until usage. Pre-treatment cancer tissues from nineteen of the patients, 16 with ACA, 2 with SCCA and 1 with adenosquamous carcinoma (ASCCA) who participated in the tissue analysis and also underwent surgery following therapy were subjected to gene expression profiling by microarray analysis.

### Histologic Evaluation

For each specimen analyzed by microarray, an adjacent tissue biopsy was given to a pathologist for assessing the presence of cancer and its histology. Routine hematoxylin-eosin-stained slides were used to evaluate for the presence of cancer in pre-treatment endoscopic biopsies and esophagectomy specimens. Post-chemoradiation resected surgical specimens with no residual cancer were classified as achieving pathCR while others with the presence of any cancer cell in the specimen were classified as less than-pathCR (<pathCR).

Synthesis of Biotin-Labeled cRNA and Hybridization

RNAs from the tissue biopsies were isolated using RNeasy® Mini kit (Qiagen, Valencia, Calif.) according to the manufacturer's recommendations. The quantity of the RNA was determined spectrophotometrically at 260 nm and the integrity of RNA was assessed by Agilent Bioanalyzer (Agilent Technologies, Palo Alto, Calif.). Only high quality RNA with intact 18S and 28S RNA was used for the synthesis of biotin labeled cRNA. As the RNA yield from some of the biopsies was less than that of 5 μg required for our standard protocol, an alternative small sample protocol with a second round of amplification that was established by the M. D. Anderson Affymetrix core facility was used to generate biotin labeled cRNA. Briefly, 200 ng of total RNA from each specimen is converted to cDNA using the Superscript choice system (Invitrogen, San Diego, Calif.), then to cRNA by in vitro transcription using Ambion MeGAscript T7 kit (Ambion, Austin, Tex.). In the second cycle, biotin-labeled cRNA is generated from the second round cDNA using the Enzo BioArray High Yield RNA Transcript Labeling Kit (Enzo Diagnostics, Farmingdale, N.Y.). The yield of biotin labeled cRNA is determined by measuring absorbance at 260 nm. Fifteen micrograms of cRNA is then fragmented and hybridized to Affymetrix U133A Chip as per manufacturer's instructions. A total of 19 RNAs isolated from pre-treatment cancer tissue from patients with ECA were subjected to microarray analysis.

### Oligonucleotide Microarray Analysis

Each U133A microarray contains 22,215 non-control probe sets that correspond to more than 18,400 distinct transcripts, including 14,593 well-characterized human genes. The list of probe sets and corresponding genes is available from the Affymetrix website. Hybridization of biotin-labeled cRNA to the oligonucleotide arrays and image analysis was performed in the DNA Microarray Core Facility at the M.D. Anderson Cancer Center according to protocols available on their website.

Microarray Suite (MAS) 5.0 software and custom tools developed by the M. D. Anderson Cancer Center Bioinformatics Department were used to analyze the data. Briefly, the microarray data were processed using PDNN model to normalize and to extract gene expression values.24 Then a hierarchical clustering algorithm was used to cluster genes and samples.25 The absent genes and the invariant genes were filtered out before clustering. The genes with below median expression value were regarded as absent genes. The invariant genes were selected according to the standard deviation of expression values across all samples (σ). The genes that have σ<three times the average of a over all the genes on the array are regarded as invariant genes. The cluster analysis was performed using the uncentered Pearson correlation as similarity metric and average linkage algorithm to combine cluster branches.

Differentially expressed genes were identified using standard t-test. The false discovery rate of the list of the differentially expressed genes was estimated using the beta-uniform mixture (BUM) distribution model.26

### Ingenuity Pathways Analysis

Ingenuity Pathways Analysis software was used to identify key functions and pathways differentially regulated between the two molecular subtypes of ECAs. The Ingenuity Pathways (INGP) Analysis software is a web-delivered application that enables biologists to discover, visualize and explore therapeutically relevant networks significant to gene expression array data sets. The INGP allows concurrent analysis of multiple datasets across different experimental platforms based on the Ingenuity Knowledge Base, a database consisting of millions of individually modeled relationships between proteins, genes, cells, tissues, drugs and diseases for the identification of key functions and pathways distinguishing biological states. A detailed description of INGP analysis is available at their website.

The average log 2 expression values were used to calculate the fold change (log 2 FC) between cancer subtypes I and II. The data set containing gene identifiers and their corresponding expression values (log 2 FC values) were then uploaded into the INGP as a tab-delimited text file to perform the analysis. Each gene identifier was mapped to its corresponding gene object in the Ingenuity Pathways Knowledge Base. A fold-change cut off of 2 was set to identify genes whose expressions were differentially regulated. These genes, called Focus Genes, were then used as the starting point for generating biological networks. To start building networks, the application queries the Ingenuity Pathways Knowledge Base for interactions between Focus Genes and all other gene objects stored in the knowledge base, and generates a set of networks with a network size of 20 genes/proteins. INGP Analysis then computes a score for each network according to the fit of the user's set of significant genes. The score is derived from a p-value and indicates the likelihood of the Focus Genes in a network being found together due to random chance. A score of 2 indicates that there is a 1 in 100 chance that the Focus Genes are together in a network due to random chance. Therefore, scores of 2 or higher have at least a 99% confidence of not being generated by random chance alone. Biological functions are then calculated and assigned to each network.

Biological functions were assigned to each gene network by using the findings that have been extracted from the scientific literature and stored in the Ingenuity Pathways Knowledge Base. The biological functions assigned to each network are ranked according to the significance of that biological function to the network. A Fischer's exact test is used to calculate a p-value determining the probability that the biological function assigned to that network is explained by chance alone.

Real Time Quantitative PCR (Real time qPCR)

To validate microarray data, the inventors chose few genes based on differential expression greater than 2 fold between the two molecular subtypes, and performed real time qPCR. cDNA for real time qPCR was generated for each sample using a kit from Invitrogen (San Diego, Calif.) according to manufacturer's instructions. Briefly, 100 ng of total RNA from the same aliquot of RNA that was used for microarray analysis was reverse transcribed using random primers and SuperScript II Reverse Transcriptase in a total volume of 20 μl. Each reaction was performed in triplicate and final reaction products were pooled and stored at −200 C until further use. The Taqman minor grove binder probe and the ABI Prism 7900 Sequence Detection system (PE Applied Biosystems, Foster City, Calif.) were used for detecting real-time PCR products. Primers and probes for the target and internal control genes were designed by Perkin Elmer Applied Biosystems and obtained via their Assays-on-Demand Gene Expression Products services. All gene expression assays have TaqMan minor groove binder probe with a corboxyfluorescein reporter dye at the 5′ end and a fluorescent quencher at the 3′-end of probe. Each target was amplified individually, PCR assays included 10 μl of Taqman universal master mix No Amperase UNG (2×), 1 μl of 20× Assays-on-Demand Gene Expression Assay Mix and 2 μl of cDNA diluted in RNase free water, in a final volume of 20 μl. The PCR thermal cycling conditions performed for all of the samples was as follows: 10 min at 95° C. for AmpliTaq Gold activation; and 40 cycles for the melting (95° C., 15 s) and annealing/extension (60° C. for 1 min) steps. PCR reactions for each target and control (18S RNA) genes were done in duplicate.

### Comparative CT Method (2^{-Δ CT}) for Relative Quantification of Gene Expression

The comparative C_{T} method was used to determine relative gene expression levels for each target gene (Livak and Schmittgen, 2001). Results of real time RT-PCR data are expressed as C_{T} values, where C_{T} is defined as the threshold PCR cycle number at which an amplified signal above the baseline is detected. There is an inverse relationship between C_{T} and amount of target, thus lower target amounts correspond to higher C_{T} and vice versa. In order to determine relative gene expression levels, first, the duplicate C_{T} values for the control (18S RNA) and the target gene were averaged for of each sample. The relative expression levels of target genes in comparison to control gene were then calculated using the formula 2^{-ΔCT} where ΔC_{T} represents the difference between each target gene and the control gene (average C_{T} for the target minus average C_{T} for 18S RNA). The relative gene expression values were multiplied by a factor of 10⁶ to make the values greater than 0.01, and to simplify presentation of the data.

### Discrimination Analysis

The potential of the three genes to discriminate the two cancer subtypes was assessed by Linear Discrimination Analysis (LDA) using S-PLUS software package (Insightful Corporation, Seattle, Wash.). The log expression values of S100A2, PERP and SPPR3 were used as predictors and labels of subtype I and subtype II were used as response variables.

TABLE 1
Patient Characteristics

| | | | | | | |
|---|---|---|---|---|---|---|
| | | | | | Baseline | |
| | | | | | Clinical | Pathologic |
| Specimen | Gender | Histology | Differentiation | Location | TNM | Outcome |

Molecular Type I

| | | | | | | |
|---|---|---|---|---|---|---|
| 1 | F | ACA | Moderate | Distal/GEJ* | T3N0M0 | CR |
| 3 | M | ACA | Moderate | Distal/GEJ | T3N1M0 | CR |
| 16 | M | ACA | Well to | Distal/GEJ | T3N1M0 | CR |
| | | | Moderate | | | |
| 23 | M | ACA | Poor | Distal Esophagus | T3N1M0 | CR |
| 56 | M | SCCA | Poor | Distal/GEJ | T3N1M0 | CR |
| 2 | M | ASCCA | Poor | Distal/GEJ | T3N0M0 | < pathCR |
| 6 | M | ACA | Moderate | Distal/GEJ | T3N1M0 | < pathCR |
| 12 | M | ACA | Poor | Distal/GEJ | T3N0M0 | < pathCR |
| 19 | F | ACA | Moderate | Distal/GEJ | T3N1M0 | < pathCR |
| 24 | F | SCCA | Poor | Mid Esophagus | T3N1M0 | < pathCR |

Molecular Type II

| | | | | | | |
|---|---|---|---|---|---|---|
| 13 | M | ACA | Moderate | Distal/GEJ | T3N1M0 | CR |
| 8 | M | ACA | Moderate | Distal Esophagus | T3N1M0 | < pathCR |
| 11 | M | ACA | Poor | Distal/GEJ | T3N1M0 | < pathCR |
| 14 | M | ACA | Moderate | Distal/GEJ | T3N1M0 | < pathCR |
| 20 | M | ACA | Moderate | Mid Esophagus | T3N1M0 | < pathCR |
| 36 | M | ACA | Poor | Distal Esophagus | T3N1M0 | < pathCR |
| 38 | M | ACA | Poor | GEJ | T3N1M0 | < pathCR |
| 44 | M | ACA | Poor | Distal/GEJ | T3N1M1a | < pathCR |
| 43 | M | ACA | Poor | Distal/GEJ | T3N0M0 | < pathCR |

### Example 2

### Gene Expression Profiling

Patient characteristics are described in Table 1.

GEJ—Gastroesophageal Junction

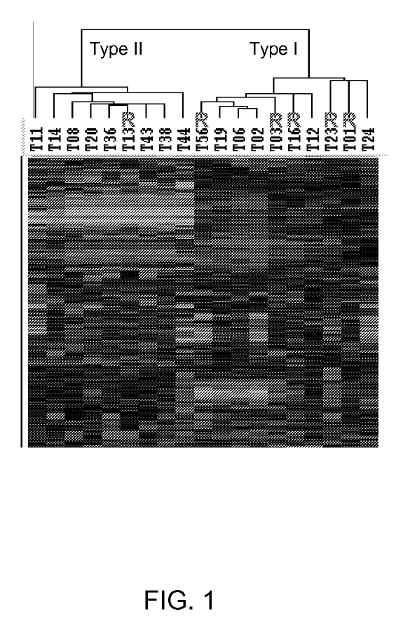

PathCR was observed in 32% (6/19) cancers. Approximately four hundred genes were differentially expressed between the two subtypes with an estimated false discovery rate of 5%. Unsupervised hierarchical cluster analysis based on these genes segregated the cancers into two major categories, each consisting of 10 and 9 cancers respectively (FIG. 1). The molecular subtype I consisted 7 ACAs and 2 SCCAs and 1 ASCCA, while subtype II consisted only ACAs. Thus, ACAs segregated into two categories. It is worth noting that the segregation of ACAs into two subtypes remained same when two of SCCAs were excluded from the clustering analysis (data not shown). Five of the cancers with pathCR (4/5 ACA and 1/1 SCCA) clustered together in type I. Subtype II, with one exception consisted cancers with <pathCR. The clustering pattern was robust against the gene filtering process and clustering algorithm used in the study. For instance, the partitioning of the subtypes remained unchanged when complete linkage algorithm was used instead of average linkage algorithm. Additionally, the partitioning of the two main sub-branches (i.e., the two subtypes) and the partitioning of the pathCR samples in to the two sub-branches remained the same when the number of variant genes included in the cluster analysis changed from 50 to 800 by altering 3σ boundary.

The median time to loco-regional and metastatic progression has not yet been reached by either of the molecular subtypes. Nevertheless, the molecular subtype II appears to portend shorter disease-free survival (DFS) time, with a mean time to DFS of 22.42 months (95% CI: 15-29) compared to 28.55 months (95% CI: 21-36) for the molecular subtype I. At 14 months 54% of subtype II was free of disease compared to 75% of subtype I. Similarly, the median time of overall survival (OS) has not been reached yet by either of the molecular subtypes. Again molecular subtype II portends a worse OS with a median OS time of 23 months (95% CI: 16-30) compared to 27.3 months (95% CI: 20-35) for subtype I. At 14 months, 57.4% of subtype II survived compared to 77.7% of subtype I.

Greater than two fold differences in the expression levels was observed in 80 genes using t-test (p<0.0001). Genes associated with apoptosis, calcium homeostasis, stress response, and proliferation were down regulated in molecular subtype II in comparison with subtype I (Table 2).

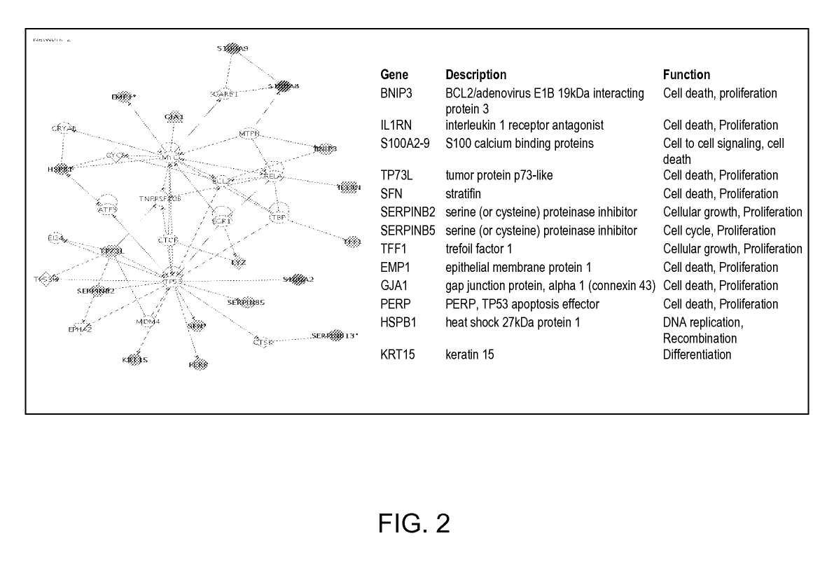

Table 2: List of genes down regulated in molecular type II cancers excluding the genes shown in FIG. 2.

TABLE 2

| Symbol | Description | Function |
|---|---|---|
| ANXA1 | Annexin A1 | Proliferation, Apoptosis |
| ANXA8 | Annexin A8 | Cell Growth, Proliferation |
| AQP3 | Aquaporin 3 | Transport |
| C1orf10, CRNN | Chromosome 1 open reading frame 10, Cornulin | Cell adehesion, Heat response |
| C1orf42 | Chromosome 1 open reading frame 42, NICE-1 protein | Differentiation |
| C4.4A | GPI-anchored metastasis-associated protein homolog | Cell-matrix interactions |
| CALML3 | Calmodulin-like 3 | Differentiation |
| CLCA | Chloride channel, calcium activated, family member 2, 4 | Transport |
| CRISP3 | Cysteine-rich secretory protein 3 | Cell-cell adhesion, Defense |
| CST | Cystatin A, B | Differentiation, CE envelope |
| DSC | Desmocollin 2, 3 | Cell Growth, Proliferation |
| DSG3 | Desmoglein 3 | Cell Growth, Proliferation |
| DSP | Desmoplakin | Embryonic Development |
| FGFBP1 | Fibroblast growth factor binding protein 1 | Cell signaling, Proliferation |
| FMO2 | Flavin containing monooxygenase 2 | Drug metabolism |
| GPR87 | G protein-coupled receptor 87 | Cell signaling |
| GPX3 | Glutathione peroxidase 3 | Oxidative damage |
| HOP | Homeodomain-only protein | Stress response |
| IVL | Involucrin | Differentiation |
| KLK1 | Kallikrein 13 | Protease |
| KRT | Keratin 4, 5, 6A, 6B, 13, 14, 15, 16, 17 | Differentiation |
| LGALS7 | Lectin, galactoside-binding, soluble, 7 | Cell-cell, Cell-matrix interactions |
| LY6D | Lymphocyte antigen 6 complex, locus D | Cell-cell adhesion |
| MAL | Mal, T-cell differentiation protein | Differentiation |
| PI3 | Protease inhibitor 3, skin-derived (SKALP) | Inflammotory response |
| PKP1 | Plakophilin 1 | Protein trafficking |
| PPL | Periplakin | Signaling |
| PPP1R3C | Protein phosphatase 1, regulatory subunit 3C | Signaling |
| RAB38 | RAB38, member RAS oncogene family | Signaling |
| RHCG | Rhesus blood group, C glycoprotein | Ammonium transport |
| SCEL | Sciellin | Differentiation, CE envelope |
| SERPINB | Serine (or cysteine) proteinase inhibitor, member 13 | Cell Growth, Proliferation |
| SERPINB4 | Serine (or cysteine) proteinase inhibitor, member 4 | Cell Growth, Proliferation |
| SPINK5 | Serine protease inhibitor, Kazal type 5 | Extracellular matrix remodeling |
| SPRR | Small proline-rich protein 1A, 1B, 2A, 2C, 3 | Differentiation, CE envelope, Proliferation |
| TACSTD2 | Tumor-associated calcium signal transducer 2 | Intracellular calcium signal |
| TGM3 | Transglutaminase 3 | CE envelope, Protein cross-linking |
| TMPRSS11E | DESC1 protein | Protease |
| TRIM29 | Tripartite motif-containing 29 | Differentiation |
| ZNF185 | Zinc finger protein 185 | Protein-protein interactions |

They include genes encoding annexin 1, chromosome 1 open reading frame 10 (C1orf10), cystatin A (GenBank® Accession No. NM_{—}005213; SEQ ID NO:17) and cystatin B (GenBank® Accession No. L03558; SEQ ID NO:18) (stefin A and B), S100 calcium binding proteins, (S100A2, S100A7 (GenBank® Accession No. NM_{—}002963; SEQ ID NO:19), S100A8, S100A9 (GenBank® Accession No. NM_{—}002965; SEQ ID NO:20), and S100A14 (GenBank® Accession No. NM_{—}020672; SEQ ID NO:21)), small proline-rich proteins (SPRR1A (GenBank® Accession No. NM_{—}005987; SEQ ID NO:24), SPRR1B (GenBank® Accession No. NM_{—}003125; SEQ ID NO:25), SPRR2A (GenBank® Accession No. NM_{—}005988; SEQ ID NO:26), SPRR2c (GenBank® Accession No. NR_{—}003062; SEQ ID NO:27), SPRR3 (GenBank® Accession No. NM_{—}005416; SEQ ID NO:28)), heat shock protein 27 (Hsp27; GenBank® Accession No. AB020027; SEQ ID NO:22)), TACSTD2 (GenBank® Accession No. NM_{—}002353; SEQ ID NO:23) and transglutaminase 3 (TGM3). Several of these proteins are Ca²⁺ binding or regulating proteins and are components of the cell envelope, which is a specialized structure that forms in terminally differentiated epithelial cells and provides a barrier against mechanical and chemical stress. For instance, TGM3, a Ca²⁺-dependent enzyme that catalyzes covalent cross-linking reactions between proteins or peptides by epsilon-gamma glutamyl lysine isopeptide bonds is important for effective epithelial barrier formation and the assembly of the cell envelope.

The top 4 functions identified by IGNP to be differentially regulated between the two molecular subtypes of ECA were: embryonic development, tissue development, cell to cell signaling and interactions, and cell death. Network profile shown in FIG. 2 generated by INGP highlights the inter-relationship between various genes and the apoptotic pathway down regulated in subtype II.

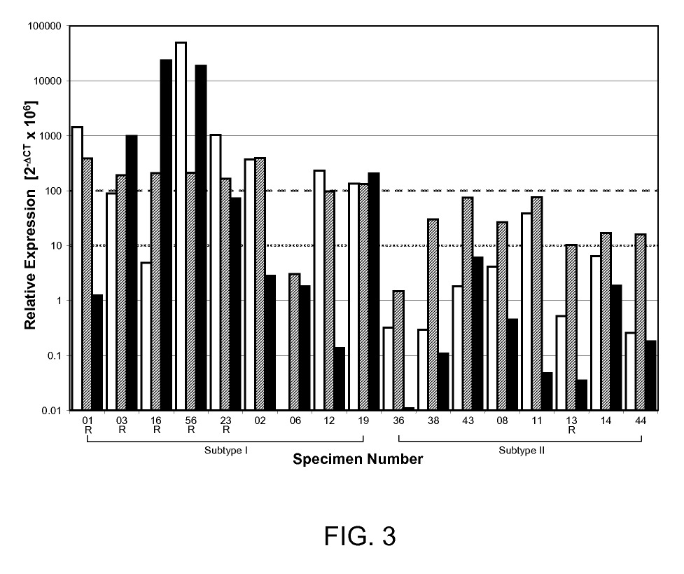

The relative expression levels of genes, PERP, S100A2 and SPRR3 evaluated by real time qPCR are shown in FIG. 3. Due to insufficient quantities of RNA, specimens 24 and 20 were not included in the real time PCR analysis. PERP is a novel type of effector involved in p53-dependent apoptosis (Ihrie et al., 2003). This protein is a member of expanding family of tetraspan membrane proteins, including PMP22 (peripheral myelin protein 22) and the epithelial membrane proteins 1, 2 and 3 (EMP1-3) (Jetten and Suter, 2000). Overexpression of EMP proteins has been shown to induce cell death through a mechanism that involves association with the P2X(7) cation channel and the consequent induction of membrane blebbing. Due to significant sequence homology to both PMP22 and EMPs and it is postulated that PERP too can induce membrane blebbing that contributes to activation of the apoptotic pathway. The S100A2 gene encoding a calcium binding protein is considered as candidate tumor-suppressor gene due to its underexpression in several cancers including esophageal SCCA in comparison to normal epithelia (Ji et al., 2004; Nagy et al., 2001; Hitomi et al., 1998). In addition, recently, S100A2 has been shown to be a novel down stream mediator of ΔNp63.33 The SPRR3, a member of small proline-rich proteins, is a component of the cell envelope and is expressed in stratified squamous epithelia during differentiation. This gene has been identified as a marker of esophageal cancer progression (Chen et al., 2000; Smolinksi et al., 2002; Kimos et al., 2004; Kimchi et al., 2005).

The relative expression values of all the three genes were lower in tumors belonging to subtype II in comparison to tumors in type I (FIG. 3) confirming our microarray data. For example, the expression values of PERP were below 75 (range 1.4-75) in subtype II, while they were over 100 (range 100-394) with one exception in cancers belonging to subtype I. Levels of S100A2 ranged between 0.3-38 and were below 10 in subtype II cancers except for cancer 11, and ranged between 5-50,000 with values above 10 in subtype I except for cancers 6 and 16. The expression of SPRR3, though overall lower in type II tumors, similarly varied among tumors ranging from 0.01 to 6 in subtype II tumors, and from 0.13 to 23,522 in subtype I. Thus, no single marker was able to segregate the two molecular subtypes without an overlap. The inventors used a statistical method (LDA) to see if the combination of genes examined by PCR have the potential to separate subtype I and subtype II as two distinct groups. Using SPRR3 and S100A2, the separation is statistically significant (p=0.014, Hotelling's T squared for differences in means between subtype I and subtype II). The p-value was 0.0006 when sample 6 that appears to be an outlier is omitted from the analysis. Thus, combining S100A2 and SPPR3 produces a classifier that separated subtype I and subtype II samples with only one outlier (sample 6).

Expression values of the three marker genes were substantially higher in cancers that achieved pathCR compared to cancer with <pathCR. When the inventors used an arbitrary cut off value of 100 for relative expression, 7/17 cancers showed expression values>100 in at least two of the three markers. These seven included five cancers that achieved pathCR (cancers 1, 3, 16, 56, and 23). Thus, only 2/17 cancers, 2 and 19, with <pathCR showed expression values>100 in at least two of the three markers. The specificity (true negatives/true negatives plus false positives) and the sensitivity (true positives/true positives plus false negatives) of the combination marker approach for identifying pathCR were 85% (11/13) and 86% (6/7) respectively.

### Example 3

### Exemplary Materials and Methods for Example 4

The present example provides exemplary materials and methods for use in certain embodiments in the invention.

### Patient Selection and Evaluation

Nineteen patients, including 16 from a previous report (Luthra et al., 2006), with localized, histologically confirmed adenocarcinoma of the thoracic esophagus were included in the study. All 19 patients participated in a clinical trial approved by The University of Texas M. D. Anderson Cancer Center's Institutional Review Board. Patients with tumors classified as T2-3 with any N, patients with Mia cancer (celiac nodes associated with a gastroesophageal junction carcinoma), and patients with T1N1 carcinoma were considered eligible. All patients were evaluated prior to registration by a multidisciplinary team that included thoracic oncology surgeons, radiation oncologists, gastroenterologists, and medical oncologists. To be eligible, patients had to have cancer that was considered technically resectable. Patients with any evidence of metastatic cancer were not enrolled.

### Treatment

The clinical protocol included 3 chemotherapy agents (docetaxel, irinotecan, and 5-fluorouracil) administered prior to and during the preoperative radiotherapy regimen. Approximately 5 to 6 weeks following the end of chemoradiotherapy, patients were restaged fully and surgery was performed if they had no metastatic cancer and no contraindications for surgery. If a patient had an R0 resection, no further therapy was planned. Patients who underwent an R1 resection (microscopic carcinoma at the margin) or R2 resection (gross carcinoma after surgery) or who had M1 disease were offered palliative care.

Each patient was assessed at 3, 6, 9, and 12 months after surgery and then every 6 months for 2 additional years and then every year or until death. Local-regional recurrence was defined as recurrence within the surgical field or mediastinal nodes. Metastatic cancer was defined as evidence of cancer outside the regional area, such as in the bone, brain, liver, or lung.

### Tissue Specimens and Collection

All tissue specimens were obtained during a diagnostic preoperative endoscopic procedure, through a protocol approved by the M. D. Anderson Cancer Center Institutional Review Board and after informed consent was obtained from patients. Both normal squamous mucosal (NSM) tissue and cancer tissue were collected from each patient. The size of a typical biopsy specimen was approximately 2.0 mm. Biopsy specimens were placed in cryogenic vials, snap frozen in liquid nitrogen, and stored at −80° C. until further use. In this report, pretreatment cancers from 19 patients and NSM tissue from 7 of these patients with high quality RNA were analyzed.

### Histologic Evaluation

For each specimen analyzed by microarray, an adjacent tissue biopsy was given to a pathologist (TTW or AR) to confirm the presence of cancer and its histology. Routine hematoxylin and eosin-stained slides were used to evaluate the presence of cancer in pretreatment endoscopic biopsies and esophagectomy specimens. The pathologic response in the resected esophagus was assigned to one of two categories: no residual carcinoma in the esophagus (pathCR) or the presence of any cancer cells in the resected specimen (<pathCR). In this cohort of patients, all except three patients underwent surgical resection and thus had pathologic response data.

### Oligonucleotide Microarray Analysis

A small sample protocol with 200 ng RNA using a second round of amplification was used to generate biotin-labeled cRNA, as described previously (Luthra et al., 2006). Fifteen micrograms of cRNA was then fragmented and hybridized to the Affymetrix U133A GeneChip (Santa Clara, Calif.) as per the manufacturer's instructions. Generation of cRNA, hybridization of biotin-labeled cRNA to the oligonucleotide arrays, and image analysis were performed according to protocols established in our institution's DNA Microarray Core Facility.

The Affymetrix U133A GeneChip contains 22,215 probe sets that correspond to 14,593 well-characterized human genes. The list of probe sets and corresponding genes is available at the Affymetrix website. Microarray Suite (MAS) 5.0 software and custom tools developed by the M. D. Anderson Bioinformatics Department were used to analyze the data. The microarray data were processed using the PDNN model to normalize and extract gene expression values (Zhang et al., 2003). The genes with below-median expression value were regarded as absent genes. The expression data of probe sets present in the 1q21-q25 region representing 517 genes were extracted and processed using an unsupervised hierarchical clustering algorithm (Eisen et al., 1998). The cluster analysis was performed using the uncentered Pearson correlation as similarity metric and average linkage algorithm to combine cluster branches. Differentially expressed genes were identified using the standard t-test.

Real-Time Quantitative Polymerase Chain Reaction (Real-Time qPCR)

We performed real-time qPCR of 9 genes that had average expression differences of twofold or greater between the NSM and cancer specimens in microarray analysis. The genes were RGS5 (regulator of G-protein signaling 5), ADAR (adenosine deaminase, RNA specific), ECM1 (extracellular matrix protein 1), IVL (involucrin), CRNN (cornulin), NICE-1, SPPR3, S100A2, and CRABP2 (cellular retinoic acid binding protein 2). Due to insufficient RNA in 5 cancers (no. 6, 20, 22, 23, and 38), the inventors were able to evaluate only 14 cancers by real-time qPCR. For cancer specimens, triplicates of 100 ng of total RNA were reverse transcribed in a final volume of 20 μl using random primers and SuperScript II™ Reverse Transcriptase (Invitrogen, Carlsbad, Calif.). The final reaction products from triplicates were pooled and stored at −20° C. until further use. For normal specimens, equal amounts of RNA from each of the 7 NSM biopsies were pooled first and then 100 ng of the pooled RNA was reverse transcribed in triplicate as described above. For both cancer and normal samples, total RNA from the same aliquot of RNA that was used for microarray analysis was used for reverse transcription.

The Taqman minor grove binder probe and the ABI Prism® 7900 HT Sequence Detection System (PE Applied Biosystems, Foster City, Calif.) were used for detecting real-time PCR products. Primers and probes for the target and internal control gene (18S) were designed by PE Applied Biosystems and obtained via their Assays-on-Demand Gene expression products services. PCR assays included 10 μl of Taqman Universal Master Mix No Amperase UNG (2×), 1 μl of 20× Assays-on-Demand Gene Expression Assay Mix, and 2 μl of cDNA diluted in Rnase-free water, in a final volume of 20 μl. The PCR thermal cycling conditions were as follows: 10 min at 95° C. for AmpliTaq Gold activation and 40 cycles for the melting (95° C., 15 s) and annealing/extension (60° C., 1 min) steps. Each target was amplified individually and in duplicate.

### Comparative CT Method (2^{-Δ CT}) for Relative Quantification of Gene Expression

The expression of each target was calculated based on the difference between amplification of the individual target mRNA template and the internal control (18S) mRNA template using the delta C_{T} (ΔC_{T}) method. The relative expression levels of target genes in comparison to the control gene were then calculated using the formula 2^{-ΔC}_{T} where ΔC_{T} represents the difference between each target gene and the control gene (average C_{T} for the target minus average C_{T} for 18S RNA). To simplify the data presentation, we multiplied 2^{-ΔC}_{T} values by a factor of 1,000,000.

### Statistical Methods

The relative expression values obtained by real-time qPCR were used to determine the potential of the genes to discriminate pathCR from <pathCR. The log expression values of IVL, CRNN, NICE-1, CRABP2, ECM1, S100A2, and SPPR3 were used as predictors, and pathCR was used as the response variable. Univariate logistic regression analysis was performed to identify markers most closely associated with pathCR.

Survival analyses were performed for overall survival (OS) and disease-free survival (DFS) times. OS time was defined as the time from registration into the trial until death from esophageal cancer. When the date of death was not available, the date of the last follow-up was used instead. Data from patients who had not died by the time of analysis were censored for the purpose of statistical analysis. DFS time was defined as the time from registration into the trial to disease recurrence or last follow-up if the date of disease recurrence or death was not available. Data from patients who were alive without disease at the time of analysis were counted as censored. The association between molecular subgroups and OS or DFS was assessed by comparing the Kaplan-Meier survival curves with the log-rank test used to test differences in survival distribution.

### Example 4

### Decreased Expression of Gene Cluster at Chromosome 1Q21

### Patient Characteristics

Table 3 illustrates the patient characteristics. Patients were mostly men, and the clinical stages were as follows: stage IIA in 16%, stage III in 74%, and stage IVA in 10%. Seven (36.8%) of the 19 patients had EAC that developed in a Barrett's esophagus background. Of the 16 patients who underwent surgical resection following chemoradiotherapy, 5 (31%) had pathCR and 11 (69%) had <pathCR (6 patients had 1-10% residual carcinoma, 2 had 11-50% residual carcinoma, and 3 had 51-100% residual carcinoma).

TABLE 3
Patient and Cancer Characteristics

| | | | | % Tumor Cells |
|---|---|---|---|---|
| | | | Barrett's | in Resected |
| Specimen | Gender | Differentiation | Association | Specimen |
| Malignant | | | | |
| Subgroup I | | | | |
| 1 | F | Moderate | NO | 0 |
| 3 | M | Moderate | NO | 0 |
| 4 | M | Moderate to poor | YES | No surgery |
| 6 | M | Moderate | NO | 1-10 |
| 12 | M | Poor | NO | 1-10 |
| 16 | M | Well to moderate | NO | 0 |
| 19 | F | Moderate | NO | 11-50 |
| 22 | M | Poor | NO | No surgery |
| 23 | M | Poor | YES | 0 |
| Malignant | | | | |
| Subgroup II | | | | |
| 8 | M | Moderate | YES | 1-10 |
| 11 | M | Poor | YES | 1-10 |
| 13 | M | Moderate | NO | 0 |
| 14 | M | Moderate | YES | 1-10 |
| 18 | M | Poor | YES | No surgery |
| 20 | M | Moderate | NO | 11-50 |
| 25/44 | M | Poor | NO | 1-10 |
| 26 | M | Poor | NO | (>50) |
| 28/38 | M | Moderate | NO | (>50) |
| 29/36 | M | Poor | YES | (>50) |

The median time to local-regional or metastatic progression was 20.5 months (range, 2 to 39 months). The median survival time was 39 months (range, 6 to 45 months), with a 3-year overall survival rate of 58%.

Molecular Subgroups by Expression Profiling of Genes at Chromosome 1q21-25

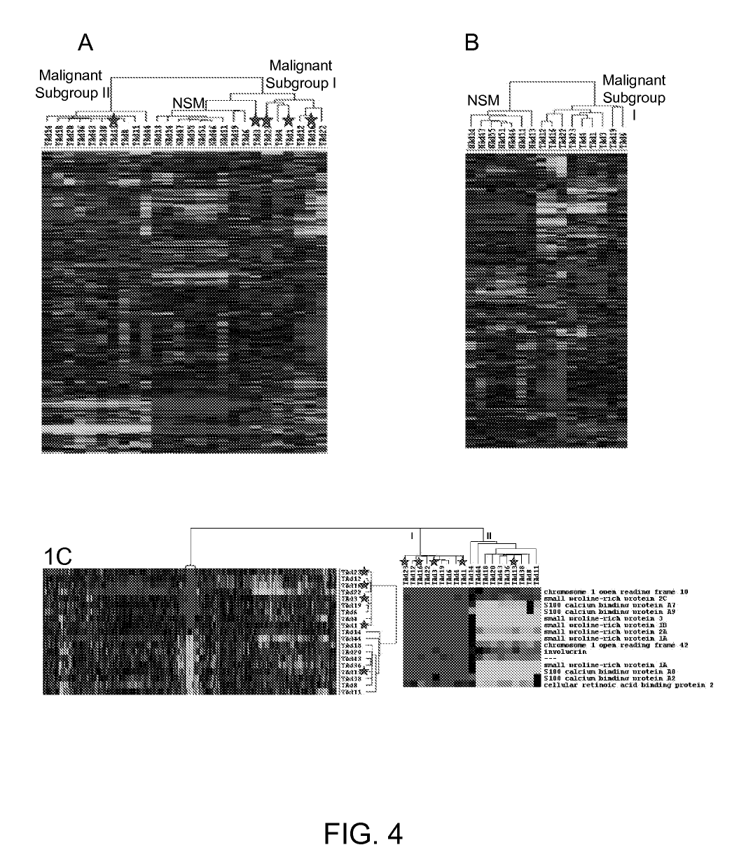

Unsupervised clustering analysis using expression data of genes mapped to the 1q21-25 chromosomal region segregated NSM specimens from cancers (FIG. 4A). Cancers, though more heterogeneous than NSM, separated into two major groups. One cluster designated as subgroup I included 9 cancers and a second cluster designated as subgroup II consisted of 10 cancers. Cancers in subgroup I demonstrated some heterogeneity but clearly segregated from NSM when subjected to cluster analysis separately without subgroup II cancers (FIG. 4B). In contrast, cancers in subgroup II clustered tightly and segregated distinctly from cancers in subgroup I, even when normal specimens were not included in the clustering analysis (FIG. 4C). It should be noted that matched NSM specimens were obtained from patients in both subgroups (3 from subgroup 1 and 4 from subgroup II cancers).

Expressional Mapping of Region 1q21-25 and Identification of Target Genes

Fifty-three genes were differentially expressed in cancer and NSM specimens (p<0.01; t-test). Table 4 shows the list of genes differentially expressed in the cancer specimens. Both cancer subgroups showed higher expression (≧twofold) of 18 genes compared to NSM.

TABLE 4
Genes differentially expressed in esophageal cancers.

| Gene Symbol | Gene Name |
|---|---|
| Downregulated | |
| in Cancers | |
| *CRNN | Cornulin (chromosome 1 open reading frame 10, |
| | C1orf10) |
| *NICE-1 | Chromosome 1 open reading frame 42, C1orf42 |
| SPRR3 | Small proline-rich protein 3 |
| *ECM1 | Extracellular matrix protein 1 |
| S100A9 | S100 calcium binding protein A9 (calgranulin B) |
| SPRR1A | Small proline-rich protein 1A |
| S100A8 | S100 calcium binding protein A8 (calgranulin A) |
| *FLG | Hypothetical gene supported by M60502 |
| SPRR2B | Small proline-rich protein 2A |
| SPRR1B | Small proline-rich protein 1B (cornifin) |
| S100A2 | S100 calcium binding protein A2 |
| *CRABP2 | Cellular retinoic acid binding protein 2 |
| RAB25 | Member RAS oncogene family |
| S100A14 | S100 calcium binding protein A14 |
| IVL | Involucrin |
| SPRR2C | Small proline-rich protein 2C |
| TUFT1 | Tuftelin 1 |
| ANXA9 | Annexin A9 |
| S100A12 | S100 calcium binding protein A12 (calgranulin C) |
| ALDH9A1 | Aldehyde dehydrogenase 9 family, member A1 |
| RIT1 | Ras-like without CAAX 1 |
| PRDX6 | Peroxiredoxin 6 |
| ENSA | Endosulfine alpha |
| FLJ11280 | Hypothetical protein FLJ11280 |
| IER5 | Immediate early response 5 |
| KIAA1614 | KIAA1614 protein |
| RASAL2 | RAS protein activator like 2 |
| YAP | YY1 associated protein |
| FCER1A | Fc fragment of IgE, high affinity I, receptor for; alpha |
| | polypeptide |
| S100A13 | S100 calcium binding protein A13 |
| HIST2H2AA | Histone 2, H2aa |
| LAD1 | Ladinin 1 |
| S100A10 | S100 calcium binding protein A10 |
| MCL1 | Myeloid cell leukemia sequence 1 (BCL2-related) |
| Upregulated | |
| in Cancers | |
| CTSS | Cathepsin S |
| JTB | Jumping translocation breakpoint |
| NDUFS2 | NADH dehydrogenase (ubiquinone) Fe—S protein 2 |
| SIP | Siah-interacting protein |
| ANP32E | Acidic (leucine-rich) nuclear phosphoprotein 32 family, |
| | member E |
| FMO5 | Flavin containing monooxygenase 5 |
| PSMB4 | Proteasome (prosome, macropain) subunit, beta type, 4 |
| CKS1B | CDC28 protein kinase regulatory subunit 1B |
| XTP2 | HBxAg transactivated protein 2 |
| IVNS1ABP | Influenza virus NS1A binding protein |
| LASS2 | LAG1 longevity assurance homolog 2 |
| TAGLN2 | Transgelin 2 |
| GPA33 | Glycoprotein A33 (transmembrane) |
| PTGS2 | Prostaglandin-endoperoxide synthase 2 |
| ADAR | Adenosine deaminase, RNA-specific |
| CTSK | Cathepsin K (pycnodysostosis) |
| RGS5 | Regulator of G-protein signaling 5 |

*Genes that show > fourfold decrease in expression compared to NSM in subgroup I.
Genes in bold indicate > fourfold decrease in expression compared to NSM in subgroup II.

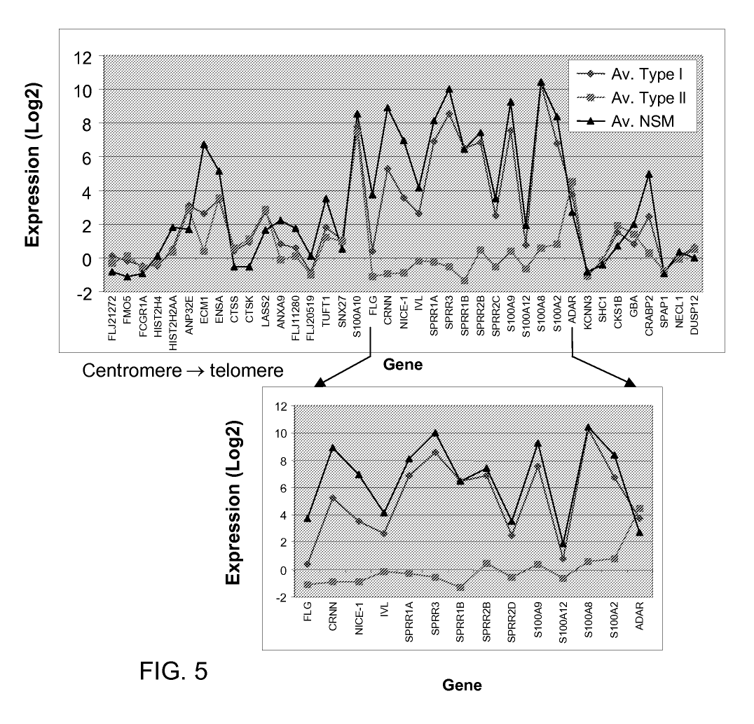

Lower expression (≧twofold) of 35 genes was seen in cancer vs. NSM specimens (FIG. 5). The expression levels of several of these genes differed substantially among cancer subgroups. For instance, 14 genes (40%) showed a greater than fourfold decrease in subgroup II cancers, whereas only 5 of the 14 genes showed such low expression in subgroup I cancers (Table 4).

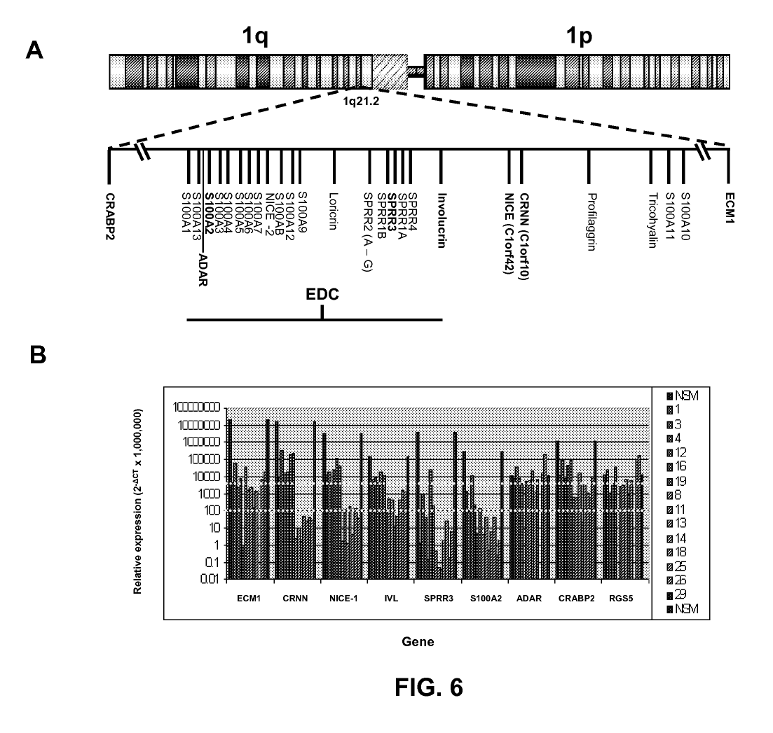

Intriguingly, within the cancer subgroups, the expression pattern suggested a gradient of decreased expression culminating with the maximal downregulation of genes included in the EDC cluster at 1q21. Thus, analysis was focused on a 5-Mb DNA region located on 1q21-q23 and encompassing the EDC genes. A schematic of the chromosomal region, with a detailed map of the EDC cluster and flanking genes, is shown in FIG. 6A. Using real-time qPCR, the level of transcriptional expression was analyzed of (1) three genes included in the EDC (IVL and SPPR3, both members of the cornified envelop precursor family, and S100A2) and (2) six genes flanking the EDC region (ECM1, ADAR, RGS5, CRNN, NICE-1, and CRABP2).

Real-time qPCR data confirmed the results obtained by microarray analysis. Compared to normal squamous epithelium, the expression levels of ECM1, IVL, CRNN, NICE-1, SPRR3, S100A2, and CRABP2 were lower in cancers, whereas the levels of ADAR and RGS5 were slightly higher in cancers.

Among cancer subgroups, subgroup II had substantially lower levels than subgroup I of expression of IVL, SPRR3, and S100A2, the three genes within the EDC (FIG. 6B). Among the genes within 0.5 Mb of the EDC cluster, the levels of CRNN and NICE-1 were also substantially lower in subgroup II compared to subgroup I. The levels of ECM1 and CRABP2, two genes flanking either side of the EDC (about 3 Mb apart), were, however, similar in both groups. A clear segregation of the two subgroups, without any overlap in the expression levels, was seen in CRNN and NICE-1. The dramatic differences in the expression levels of CRNN, NICE-1, IVL, SPRR3, and S100A2 segregated the cancers into high (subgroup I) and low expressers (subgroup II). It should be noted that substantial heterogeneity was found in gene expression levels among the cancers in both subgroups.

It has been suggested that within the 1q21 region, the EDC gene cluster and its closely flanking genes may have a coordinated transcription control mechanism (Zhao and Elder, 1997; Mischke, 1998; Williams et al., 2002). Hence, using the data obtained by real-time qPCR, the correlation between the expression levels of 1q21 target genes using linear relationship analysis methods was measured.

Table 5: Linear correlation analysis of log expression levels showing coordinate regulation of target genes.

TABLE 5

| Gene Name | SPRR3 | S100A2 | CRNN | NICE-1 | CRABP2 | ECM1 | IVL |
|---|---|---|---|---|---|---|---|
| SPRR3 | 1.00000 | 0.31056 | 0.75039 | 0.57437 | 0.49195 | −0.41611 | 0.35021 |
| | | 0.3017 | 0.0031 | 0.0401 | 0.0877 | 0.1573 | 0.2408 |
| S100A2 | 0.31056 | 1.00000 | 0.67695 | 0.58679 | 0.51717 | 0.07853 | 0.40646 |
| | 0.3017 | | 0.0110 | 0.0350 | 0.0703 | 0.7987 | 0.1681 |
| CRNN | 0.75039 | 0.67695 | 1.00000 | 0.82184 | 0.57673 | −0.04184 | 0.51497 |
| | 0.0031 | 0.0110 | | 0.0006 | 0.0391 | 0.8920 | 0.0717 |
| NICE-1 | 0.57437 | 0.58679 | 0.82184 | 1.00000 | 0.69831 | 0.08612 | 0.61769 |
| | 0.0401 | 0.0350 | 0.0006 | | 0.0079 | 0.7797 | 0.0245 |
| CRABP2 | 0.49195 | 0.51717 | 0.57673 | 0.69831 | 1.00000 | −0.03677 | 0.80557 |
| | 0.0877 | 0.0703 | 0.0391 | 0.0079 | | 0.9051 | 0.0009 |
| ECM1 | −0.41611 | 0.07853 | −0.04184 | 0.08612 | −0.03677 | 1.00000 | −0.04950 |
| | 0.1573 | 0.7987 | 0.8920 | 0.7797 | 0.9051 | | 0.8724 |
| IVL | 0.35021 | 0.40646 | 0.51497 | 0.61769 | 0.80557 | −0.04950 | 1.00000 |
| | 0.2408 | 0.1681 | 0.0717 | 0.0245 | 0.0009 | 0.8724 | |

Statistically significant correlation is seen between CRNN and NICE-1, S100A2, SPRR3, CRABP2, and IVL and between CRABP2 and CRNN, NICE-1, and IVL, as indicated by p values shown in bold.

Strikingly, as shown in Table 5, there was a statistically significant coordinated downregulation of the EDC genes and their neighboring genes, CRNN, NICE-1, and CRABP2.

### Molecular Malignant Subgroups, Clinical Characteristics, and Patient Outcome

The 1q21-25 molecular malignant subgroups were not associated with patient characteristics, including age at diagnosis, clinical stage, and location of the primary tumor. It is noteworthy that 5 (71%) of the 7 patients with EAC that developed in the background of Barrett's esophagus were in subgroup II (Table 3).

The molecular malignant subgroups appeared to be associated with response to CTXRT. Four (80%) of the 5 patients achieving pathCR were clustered in molecular subgroup I compared to only 1 patient (20%) in subgroup II, suggesting that subgroup II cancers were resistant to CTXRT. While expression levels of CRNN, NICE-1, IVL, SPRR3, and S100A2 were substantially different in the two EAC subgroups (FIG. 6), a statistically significant relationship between these biomarkers and pathCR was not shown by univariate logistic regression analysis. Three biomarkers, IVL, NICE-1, and CRNN, on the basis of minimal overlap in the expression levels between the two subgroups of cancers, were dichotomized into high/low expression (high expression was defined as relative expression levels of >100 for CRNN and >5000 for NICE-1 and IVL). Then, a two-sided Fisher's exact test was used to investigate the association of the dichotomized biomarkers with pathologic complete response. A statistically significant association with pathCR was observed only for IVL (P=0.05). However, the small sample size may have underpowered the study. Due to the heterogeneity in the expression levels of genes within the EDC, it was not possible to segregate responders from nonresponders within subtypes.

Patients in molecular subgroup II had worse OS, with a median time of 34 months (95% CI: 16-52 months) compared to 44 months (95% CI: 0-90 months) for those in subgroup I; however, owing to the small cohort size, no statistically significant difference was reached (P=0.55; log-rank test). At 3 years, 32% of subgroup II survived compared to 78% of subgroup I. Similarly, molecular subgroup II was associated with shorter DFS, with a median DFS time of 14 months (95% CI: 2-26 months) compared to 37 months (95% CI: 0-82 months) for molecular subgroup I (P=0.20; log-rank test). At 3 years, 44% of patients in subgroup II were disease-free compared to 78% in subgroup I.

### Example 5

### Significance of the Present Invention

The clinical course of patients with ECA is heterogeneous. Thus patients with the same disease stage have variable outcomes from uniform therapy. Patients with chemoradiation-resistant cancer have a very high likelihood of developing metastases (Rohatgi et al., 2005). Currently, an empiric approach is utilized for patients with local-regional esophageal cancer, since one is not able to predict the degree of chemoradiation-resistance prior to surgery. The early identification of non-responders would allow physicians to discontinue ineffective treatment regimens and institute alternative treatments, thereby avoiding both over-treatment and under-treatment of patients. Therefore, the need for markers that predicts response early during the course of therapy is widely acknowledged.

In an attempt to identify a panel of biomarkers that allow us to predict response to chemoradiation, we profiled pre-treatment cancer biopsies from 19 patients enrolled in a clinical protocol. Six (32%) of these patients had a pathCR. Unsupervised cluster analysis separated the cancers into two categories. Interestingly, five out of the six cancers (83%) that achieved pathCR clustered in one molecular subtype (type I). Only one cancer with pathCR fell in subtype II.

There was no clear segregation, however, of pathCR from <pathCR in subtype I, as 30% (5/13) of cancers with <pathCR also clustered in this subtype. Nevertheless, the PCR data point out that expression analysis of a limited set of biomarkers selected from the list of genes that were differentially regulated between the two subtypes increases the predictive power. Thus, simply using three markers, PERP, S100A2 and SPRR3, and choosing an arbitrary expression cutoff value of 100, the inventors were able to assign cancers to pathCR and <pathCR categories in 15/17 cancers tested by PCR.

Median time to loco-regional and metastatic progression were not reached by either of the molecular subtypes. Similarly, the median time of overall survival (OS) was not reached yet by either of the molecular subtypes. However, the molecular subtype II appears to portend shorter disease free survival (DFS) time, and a worse OS compared to subtype I.

Many of the genes with differential expression between the two types of ECAs have been previously reported to show altered expression in esophageal cancers by other investigators confirming that they were cancer related (Dahlberg et al., 2004; Luo et al., 2004; Ji et al., 2004; Kimos et al., 2004; Abraham et al., 1996; Soldes et al., 1999; Soldes et al., 1999; Doak et al., 2004; Paweletz et al., 2000; Shiraishi et al., 1998). It is interesting to note that Luo et al., 21 using high-density cDNA microarray platform, also observed that several genes including annexin 1, SPRRS, S100A8 and A9, TGM3, CK4, CK13, and CK15, were down regulated in SCCA in comparison to normal squamous epithelium.

Collective down regulation of several members of apoptotic pathway such Bcl-2/EIB 19 kDa interacting protein 3 (BNIP3), PERP, epithelial membrane protein (EMP1), p63, stratifin (SFN)/14-3-3σ and S100A2 in non-responder cancer type as illustrated in network profile (FIG. 2) implicates a critical role of apoptotic pathway in chemoradiation resistance in ECA. Solid tumors are poorly oxygenated as compared with normal tissues and consist regions of hypoxia. These hypoxic regions often correlate with poor prognosis due to the ability of cells within these regions to become resistant to chemotherapeutic reagents and radiation therapy. Apoptosis induced by hypoxia is a mechanism for elimination of stressed cells. Similarly, ionizing radiation and chemotherapeutic agents use the process of programmed cell death to induce cancer cell death. In vitro studies have shown that several genes we noticed to be differentially regulated between the two molecular types were indeed associated with response to chemoradiation. For example, BNIP3 encoded by Bnip3L, a unique member of the Bcl2 family members is down regulated in cancer cells that are resistant to the 5-FU.44 Vande Velde et al. 45 have shown that forced overexpression of BNIP3 induces cell death characterized by localization at the mitochondria, loss of membrane potential and reactive oxygen species (ROS) production. More recent studies have demonstrated that Bnip3L is inducible by p53 under hypoxia and its knockdown promotes tumor growth.46 Similarly, Hermeking et al. (1997), have demonstrated that SFN is induced after DNA damage in a p53 dependent manner. It is also shown to play a crucial role in the G2 checkpoint by sequestering the mitotic initiation complex, cdc2-cyclin B1, in the cytoplasm after DNA damage (Chan et al., 2000). Further, upon cisplatin induced DNA damage, SFN is shown to bind with phosphorylated ΔNp63α isoform and mediate nuclear export of ΔNp63α into cytoplasm (Fomenkov et al., 2004).

This is the first report showing two types of esophageal ACA with distinct molecular signatures. It is clear from published studies that the genes differentially expressed in the two molecular subtypes in our study are cancer related genes. Since many of these genes are highly and uniformly expressed in normal squamous epithelium, earlier profiling studies comparing tumors to normal squamous epithelium may have clustered tumors with varying degree of loss of expression in to one category. Excluding normal esophageal mucosa in microarray analysis in our strategy may in fact have accentuated the separation of the molecular subtypes based on differences in the relative expression levels among the tumors and not between tumors and normal mucosa. Thus, it appears that it is not the loss or gain of expression of these genes in comparison to normal squamous epithelium, but it is the relative levels in different tumors that distinguish responders from non-responders. As our tumor specimens were unselected with regard to percentage of stromal infiltration or inflammation, we realize that the clustering results might reflect contributions from non-neoplastic cellular elements to the expression signatures. However, both tumor and its surrounding microenvironment are important in tumor growth and response, inclusion of these components may be beneficial than detrimental in studies such as ours that are designed to associate molecular signatures with pathologic response. The data indicate that analysis of combination of biomarkers that are easily analyzed by quantitative assays such as PCR may be sufficient for distinguishing cancers that respond to therapy from those resistant to therapy. In specific embodiments, the studies are repeated with a larger set of samples to characterize the predictive power of these markers.

Chemoradiation resistance and local recurrence or distant metastases are clinical features of over 75% of esophageal cancers treated with CTXRT. Thus, understanding the biological properties that render a cancer sensitive or insensitive to chemoradiation is crucial for tumor management.

To begin to understand biomarkers predictive of response to CTXRT, the inventors previously conducted a transcriptional profiling study of pretreatment esophageal cancer biopsies derived from patients treated on a preoperative CTXRT protocol. Among the markers associated with lack of response to CTXRT, there was downregulation of genes located at the chromosomal region 1q21. The present analysis of the 517 genes within the 1q21-25 region clearly shows EAC resistant to CTXRT clustered together with a homogenous down-regulation of genes in this region compared with EAC sensitive to CTXRT.

The in-depth analysis of the genes included in the 1q21-25 region suggests a gradient of expression changes between cancer molecular subgroups. EAC sensitive to CTXRT harbored a transcriptional profile closer to that of NSM, whereas resistant EAC showed a dramatically different profile stemming mainly from the differential expression of the EDC gene cluster, including CRNN, NICE-1, IVL, SPRR3, and S100A2 (FIGS. 6A and 6B). Relative expression levels of these genes, as determined by quantitative PCR, clearly segregate the two cancer types.

The proteins encoded within the EDC are implicated in the terminal differentiation of keratinocytes, and their expression is temporarily physiologically coordinated to mediate cessation of proliferation (e.g., S100As) and migration toward the superficial layers (e.g., IVL), with associated progressive cornification (e.g., SPRRs). The temporal expression of these proteins appears to be a biological program, coordinated by the subchromosomal position of gene territories and similar to that involving the major histocompatibility complex genes (Williams et al., 2002). The EDC cluster is also critical for the maturation and maintenance of the stratified squamous normal esophageal mucosa. While the functions of the proteins encoded by the NICE-1, IVL, SPRR3, and S100A2 genes in terminally differentiating keratinocytes are well documented, the role of CRNN in the differentiation program is poorly understood. CRNN is either dramatically reduced or absent in primary esophageal cancer tissues, suggesting that it is esophageal specific and cancer related (Contzier et al., 2005; Xu et al., 2000). The data, along with a previous report by Kimchi et al (2005), imply that the downregulation of genes in this chromosomal region is involved in EAC development and progression. The report also demonstrates an association between these genes and CTXRT response.

There are some key observations stemming from certain embodiments of the present invention. These include (1) the degree of transcriptional suppression of genes mapping within and close to the EDC can segregate EAC into low and high expressers; (2) the high expressers, while showing transcriptional suppression in the EDC cluster region, appear to have a transcriptional signature closer to that of NSM; (3) the high expressers are more likely to be sensitive to CTXRT; and (4) the majority of the cancers with associated Barrett's metaplasia cluster in CTXRT-resistant cancer subgroup II. Thus, these observations indicate that EAC may include biologically and molecularly different entities: subgroup I maintaining similarity to NSM and subgroup II having a molecular signature similar to glandular epithelium. Of importance, cancers within subgroup I were more sensitive to CTXRT, similar to esophageal squamous cell carcinoma, for which the 3-year survival rate after CTXRT is higher than that for EAC (Naughton and Walsh, 2004; Varadhachary and Hoff, 2005), and cancers within subgroup II were resistant to CTXRT, similar to EAC associated with Barrett's metaplasia (Agarwal et al., 2005). In one embodiment, EAC associated with Barrett's metaplasia is more resistant to chemoradiation. Additional studies may be performed to further characterize which molecular pathways are important for imparting chemoradiation resistance in subgroup II.

Cellular retinoic acid-binding protein II (CRABP-II), encoded by CRABP2, is an intracellular lipid-binding protein that associates with retinoic acid with a subnanomolar affinity. Studies have shown that retinoic acid regulates expression of markers of differentiation (Hohl et al., 1995; Asselineau et al., 1989; Hohl et al., 1991). Since it has been reported that CRABP-II enhances the transcriptional activity of the nuclear receptor, the retinoic acid receptor (RAR), by delivering retinoic acid to this receptor, the retinoic acid pathway may be affected by decreased expression of CRABP2 in these cancers.

The results, along with those of other investigators, provide independent confirmation that EDC genes are differentially regulated in EAC and NSM and validate these genes as markers of EAC. The present invention is the first to identify coordinated downregulation of keratinocyte differentiation genes and CRABP2 and the association between transcriptional suppression of differentiation-associated genes and resistance to chemoradiation in EAC. Studies using a larger sample set are in progress to validate IVL, CRNN, and NICE-1, in addition to previously shown SPRR3 and S100A2, as markers of resistance to CTXRT in EAC.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### Patents

### Publications

Abraham J M, Wang S, Suzuki H, et al: Esophagin cDNA cloning and characterization: a tissue-specific member of the small proline-rich protein family that is not expressed in esophageal tumors. Cell Growth Differ 7:855-60, 1996
Berger A C, Farma J, Scott W J, et al: Complete Response to Neoadjuvant Chemoradiotherapy in Esophageal Carcinoma Is Associated With Significantly Improved Survival. J Clin Oncol, 2005
Bollschweiler E, Wolfgarten E, Gutschow C, et al: Demographic variations in the rising incidence of esophageal adenocarcinoma in white males. Cancer 92:549-55, 2001
Brabender J, Marjoram P, Salonga D, et al: A multigene expression panel for the molecular diagnosis of Barrett's esophagus and Barrett's adenocarcinoma of the esophagus. Oncogene 23:4780-8, 2004
Chan T A, Hwang P M, Hermeking H, et al: Cooperative effects of genes controlling the G(2)/M checkpoint. Genes Dev 14:1584-8, 2000
Chen B S, Wang M R, Cai Y, et al: Decreased expression of SPRR3 in Chinese human oesophageal cancer. Carcinogenesis 21:2147-50, 2000
Chirieac L R, Swisher S G, Ajani J A, et al: Posttherapy pathologic stage predicts survival in patients with esophageal carcinoma receiving preoperative chemoradiation. Cancer 103:1347-55, 2005
Dahlberg P S, Ferrin L F, Grindle S M, et al: Gene expression profiles in esophageal adenocarcinoma. Ann Thorac Surg 77:1008-15, 2004
Darnton S J, Archer V R, Stocken D D, et al: Preoperative mitomycin, ifosfamide, and cisplatin followed by esophagectomy in squamous cell carcinoma of the esophagus: pathologic complete response induced by chemotherapy leads to long-term survival. J Clin Oncol 21:4009-15, 2003
de Angelis P M, Fjell B, Kravik K L, et al: Molecular characterizations of derivatives of HCT116 colorectal cancer cells that are resistant to the chemotherapeutic agent 5-fluorouracil. Int J Oncol 24:1279-88, 2004
Doak S H, Jenkins G J, Parry E M, et al: Differential expression of the MAD2, BUB1 and HSP27 genes in Barrett's oesophagus-their association with aneuploidy and neoplastic progression. Mutat Res 547:133-44, 2004
Eisen M B, Spellman P T, Brown P O, et al: Cluster analysis and display of genome-wide expression patterns. Proc Natl Acad Sci USA 95:14863-8, 1998
Enzinger P C, Mayer R J: Esophageal cancer. N Engl J Med 349:2241-52, 2003.
Fei P, Wang W, Kim S H, et al: Bnip3L is induced by p53 under hypoxia, and its knockdown promotes tumor growth. Cancer Cell 6:597-609, 2004
Fomenkov A, Zangen R, Huang Y P, et al: RACK1 and stratifin target DeltaNp63alpha for a proteasome degradation in head and neck squamous cell carcinoma cells upon DNA damage. Cell Cycle 3:1285-95, 2004
Hermeking H, Lengauer C, Polyak K, et al: 14-3-3 sigma is a p53-regulated inhibitor of G2/M progression. Mol Cell 1:3-11, 1997
Hibi K, Fujitake S, Takase T, et al: Identification of S100A2 as a target of the DeltaNp63 oncogenic pathway. Clin Cancer Res 9:4282-5, 2003
Hitomi J, Kimura T, Kusumi E, et al: Novel S100 proteins in human esophageal epithelial cells: CAAF1 expression is associated with cell growth arrest. Arch Histol Cytol 61:163-78, 1998
Hourihan R N, O'Sullivan G C, Morgan J G: Transcriptional gene expression profiles of oesophageal adenocarcinoma and normal oesophageal tissues. Anticancer Res 23:161-5, 2003
Ihrie R A, Reczek E, Horner J S, et al: Perp is a mediator of p53-dependent apoptosis in diverse cell types. Curr Biol 13:1985-90, 2003
Ilson D H: Oesophageal cancer: new developments in systemic therapy. Cancer Treat Rev 29:525-32, 2003
Ishibashi Y, Hanyu N, Nakada K, et al: Profiling gene expression ratios of paired cancerous and normal tissue predicts relapse of esophageal squamous cell carcinoma. Cancer Res 63:5159-64, 2003
Jemal A, Murray T, Samuels A, et al: Cancer statistics, 2003. CA Cancer J Clin 53:5-26, 2003
Jetten A M, Suter U: The peripheral myelin protein 22 and epithelial membrane protein family. Prog Nucleic Acid Res Mol Biol 64:97-129, 2000
Ji J, Zhao L, Wang X, et al: Differential expression of S100 gene family in human esophageal squamous cell carcinoma. J Cancer Res Clin Oncol 130:480-6, 2004
Kazemi-Noureini S, Colonna-Romano S, Ziaee A A, et al: Differential gene expression between squamous cell carcinoma of esophagus and its normal epithelium; altered pattern of mal, akr1c2, and rab11a expression. World J Gastroenterol 10:1716-21, 2004
Kimchi E T, Posner M C, Park J O, et al: Progression of Barrett's metaplasia to adenocarcinoma is associated with the suppression of the transcriptional programs of epidermal differentiation. Cancer Res 65:3146-54, 2005
Kimos M C, Wang S, Borkowski A, et al: Esophagin and proliferating cell nuclear antigen (PCNA) are biomarkers of human esophageal neoplastic progression. Int J Cancer 111:415-7, 2004
Livak K J, Schmittgen T D: Analysis of relative gene expression data using real-time quantitative PCR and the 2(-Delta Delta C(T)) Method. Methods 25:402-8, 2001
Lu J, Liu Z, Xiong M, et al: Gene expression profile changes in initiation and progression of squamous cell carcinoma of esophagus. Int J Cancer 91:288-94, 2001
Luo A, Kong J, Hu G, et al: Discovery of Ca2+-relevant and differentiation-associated genes downregulated in esophageal squamous cell carcinoma using cDNA microarray. Oncogene 23:1291-9, 2004
McManus D T, Olaru A, Meltzer S J: Biomarkers of esophageal adenocarcinoma and Barrett's esophagus. Cancer Res 64:1561-9, 2004
Nagy N, Brenner C, Markadieu N, et al: S100A2, a putative tumor suppressor gene, regulates in vitro squamous cell carcinoma migration. Lab Invest 81:599-612, 2001
Paweletz C P, Ornstein D K, Roth M J, et al: Loss of annexin 1 correlates with early onset of tumorigenesis in esophageal and prostate carcinoma. Cancer Res 60:6293-7, 2000
Pera M: Recent changes in the epidemiology of esophageal cancer. Surg Oncol 10:81-90, 2001
Pera M: Trends in incidence and prevalence of specialized intestinal metaplasia, barrett's esophagus, and adenocarcinoma of the gastroesophageal junction. World J Surg 27:999-1008; discussion 1006-8, 2003
Pounds S, Morris S W: Estimating the occurrence of false positives and false negatives in microarray studies by approximating and partitioning the empirical distribution of p-values. Bioinformatics 19:1236-42, 2003
Rohatgi P, Swisher S G, Correa A M, et al: Association of pathologic complete response after preoperative chemoradiotherapy in carcinoma of the esophagus and outcome after pathologic complete response. Cancer In Press, 2005
Shiraishi T, Mori M, Tanaka S, et al: Identification of cystatin B in human esophageal carcinoma, using differential displays in which the gene expression is related to lymph-node metastasis. Int J Cancer 79:175-8, 1998
Smolinski K N, Abraham J M, Souza R F, et al: Activation of the esophagin promoter during esophageal epithelial cell differentiation. Genomics 79:875-80, 2002
Soldes O S, Kuick R D, Thompson I A, 2nd, et al: Differential expression of Hsp27 in normal oesophagus, Barrett's metaplasia and oesophageal adenocarcinomas. Br J Cancer 79:595-603, 1999
Vande Velde C, Cizeau J, Dubik D, et al: BNIP3 and genetic control of necrosis-like cell death through the mitochondrial permeability transition pore. Mol Cell Biol 20:5454-68, 2000
Wild C P, Hardie L J: Reflux, Barrett's oesophagus and adenocarcinoma: burning questions. Nat Rev Cancer 3:676-84, 2003
Winters C, Jr., Spurling T J, Chobanian S J, et al: Barrett's esophagus. A prevalent, occult complication of gastroesophageal reflux disease. Gastroenterology 92:118-24, 1987
Xu S H, Qian L J, Mou H Z, et al: Difference of gene expression profiles between esophageal carcinoma and its pericancerous epithelium by gene chip. World J Gastroenterol 9:417-22, 2003
Xu Y, Selaru F M, Yin J, et al: Artificial neural networks and gene filtering distinguish between global gene expression profiles of Barrett's esophagus and esophageal cancer. Cancer Res 62:3493-7, 2002
Zhang L, Miles M F, Aldape K D: A model of molecular interactions on short oligonucleotide microarrays. Nat Biotechnol 21:818-21, 2003
Zhou J, Zhao L Q, Xiong M M, et al: Gene expression profiles at different stages of human esophageal squamous cell carcinoma. World J Gastroenterol 9:9-15, 2003

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of determining effectiveness of a cancer therapy in an individual with cancer, comprising assaying gene expression levels in a sample from the individual, wherein said sample comprises RNA, protein, or both, wherein said assaying comprises determining the expression of one or more esophageal cancer-associated genes selected from the group consisting of SEQ ID NO:1, SEQ ID NO:2, SEQ ID NO:3, SEQ ID NO:4, SEQ ID NO:5, and SEQ ID NO:6.

2. (canceled)

3. (canceled)

4. The method of claim 1, further defined as discriminating whether or not the individual will have pathologic complete response or less than pathologic complete response.

5. (canceled)

6. (canceled)

7. The method of claim 1, wherein said assaying comprises determining the expression level of two or more, or three or more, or four or more genes selected from the group consisting of SEQ ID NO:1, SEQ ID NO:2, SEQ ID NO:3, SEQ ID NO:4, SEQ ID NO:5, and SEQ ID NO:6.

8. (canceled)

9. (canceled)

10. (canceled)

11. (canceled)

12. The method of claim 1, wherein said assaying comprises subjecting a substrate having nucleic acids affixed thereto to RNA from the sample.

13. The method of claim 1, wherein said assaying comprises subjecting cDNA from the RNA from the sample to polymerase chain reaction.

14. The method of claim 1, wherein said assaying comprises subjecting one or more antibodies to proteins from the sample.

15. The method of claim 1, wherein the cancer therapy comprises chemotherapy, radiation, surgery, or a combination thereof.

16. The method of claim 1, wherein said method further comprises obtaining the sample from the individual.

17. The method of claim 16, wherein said obtaining of the sample comprises biopsy, obtaining saliva, obtaining gastric juice, or a combination thereof.

18. The method of claim 12, wherein one or more nucleic acids affixed to the substrate anneal under stringent conditions to at least two of, at least three of, at least four of, or at least five of the sequences in the RNA of the sample, said sequences selected from the group consisting of SEQ ID NO:1, SEQ ID NO:2, SEQ ID NO:3, SEQ ID NO:4, SEQ ID NO:5, and SEQ ID NO:6.

19. (canceled)

20. (canceled)

21. (canceled)

22. The method of claim 12, wherein one or more nucleic acids affixed to the substrate anneal under stringent conditions to all of the sequences in the RNA of the sample, said sequences selected from the group consisting of SEQ ID NO:1, SEQ ID NO:2, SEQ ID NO:3, SEQ ID NO:4, SEQ ID NO:5, and SEQ ID NO:6.

23. The method of claim 12, wherein at least 10%, at least 20%, at least 30%, at least 40%, at least 50%, at least 60%, at least 70%, at least 80%, or at least 90% of the nucleic acids on the substrate are capable of hybridizing under stringent conditions to RNA in the sample.

24. (canceled)

25. (canceled)

26. (canceled)

27. (canceled)

28. (canceled)

29. (canceled)

30. (canceled)

31. (canceled)

32. The method of claim 12, wherein substantially all of the nucleic acids on the substrate are capable of hybridizing under stringent conditions to RNA in the sample.

33. The method of claim 1, further comprising subjecting the individual to x-ray, barium swallow, biopsy, esophagoscopy, or a combination thereof.

34. The method of claim 1, wherein when the effectiveness of the cancer therapy is determined to be non-effective, the individual is provided with an alternative therapy.

35. The method of claim 34, wherein the alternative therapy comprises surgery, chemotherapy, radiation, or a combination thereof.

36. An isolated substrate, comprising two or more of polynucleotides affixed thereto, wherein said polynucleotides are selected from the group consisting of SEQ ID NO:1, SEQ ID NO:2, SEQ ID NO:3, SEQ ID NO:4, SEQ ID NO:5, and SEQ ID NO:6.

37. A plurality of primers suitable for use in amplifying one or more of the polynucleotides are selected from the group consisting of SEQ ID NO:1, SEQ ID NO:2, SEQ ID NO:3, SEQ ID NO:4, SEQ ID NO:5, and SEQ ID NO:6.

38. (canceled)

39. (canceled)

40. (canceled)

41. (canceled)

42. (canceled)

43. (canceled)
